(19) United States
(12) Patent Application Publication
(10) Pub. No.: US 2011/0021524 A1
(43) Pub. Date: Jan. 27, 2011

(54) COMPOSITIONS AND METHODS FOR TREATING CANCERS

(75) Inventors: Francisco Adrian, San Diego, CA (US); Christine Dierks, Freiburg (DE); Nathanael S. Gray, Boston, MA (US); Markus Warmuth, Natick, MA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/812,740

(22) PCT Filed: Dec. 18, 2008
(86) PCT No.: PCT/US08/87344

§ 371 Date: Oct. 11, 2010

Related U.S. Application Data
(60) Provisional application No. 61/021,008, filed on Jan. 14, 2008.

(51) Int. Cl. A61K 31/5377; A61K 31/506; A61K 31/505; A61K 31/52; A61K 31/497; A61K 31/5025; A61P 35/02; A61P 35/00

(52) U.S. Cl. 514/235.8; 514/275; 514/263.4; 514/252.16; 514/257; 514/252.18; 514/252.19

(57) ABSTRACT

This invention provides a combination of ATP-competitive BCR-ABL inhibitor and a non-ATP competitive BCR-ABL inhibitor. The combination of the present invention may be used for treating cancers known to be associated with BCR-ABL.

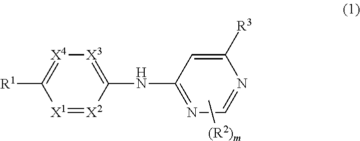

### CROSS-REFERENCE TO RELATED APPLICATIONS

This application claims the benefit of U.S. provisional application Ser. No. 61/021,008, filed 14 Jan. 2008, which is hereby incorporated by reference in its entirety.

### TECHNICAL FIELD

The present invention generally relates to combination therapy and methods for inhibiting tumor cell growth and for treating cancer.

### BACKGROUND ART

The BCR-ABL oncogene is the product of Philadelphia chromosome (Ph) 22q, and encodes a chimeric BCR-ABL protein that has constitutively activated ABL tyrosine kinase activity. (Lugo et al., Science 247:1079-1082 (1990)). BCR-ABL is the underlying cause of chronic myeloid leukemia. Whereas the 210 kDa BCR-ABL protein is expressed in patients with CML, a 190 kDa BCR-ABL protein resulting from an alternative breakpoint in the BCR gene is expressed in patients with Ph positive (Ph⁺) acute lymphoblastic leukemia (ALL). (Bartram et al., Nature 306:277-280 (1983); Chan et al., Nature 325:635-637 (1987)). BCR-ABL has been shown to induce proliferation and anti-apoptosis through various mechanisms in committed myeloid or lymphoid progenitors or 3T3 fibroblasts. (Pendergast et al., Cell 75:175-85 (1993); Ilaria et al., J. Biol. Chem. 271:31704-10 (1996); Chai et al., J. Immunol. 159:4720-8 (1997); and Skorski et al., EMBO J. 16:6151-61 (1997)).

### DISCLOSURE OF THE INVENTION

The invention provides compositions, particularly combination therapy, which may be useful for inhibiting tumor cell growth and for treating a variety of cancers.

In one aspect, the invention provides a composition comprising an ATP-competitive BCR-ABL inhibitor and a non-ATP competitive BCR-ABL inhibitor;

wherein the ATP-competitive inhibitor is selected from the group consisting of imatinib (STI571), nilotinib (AMN107), pyrido[2,3-d]pyrimidine compounds (e.g., dasatinib), bosutinib, 3-substituted benzamide derivatives (e.g., INNO-406), AZD-0530, MK-0457, PHA-739358, AP24534 (Ariad), JNJ-26483327(Johnson & Johnson), HPK-61 (SuperGen), SKS-927 (Wyeth), AT-9283 (Astex Pharmaceuticals), EXEL-2280 (Exelisis) and TG-100572 (Targegen); and

wherein said ATP non-competitive BCR-ABL inhibitor is a compound of Formula (1):

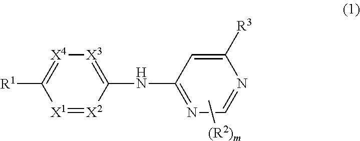

or a pharmaceutically acceptable salt thereof;

wherein X¹, X², X³ and X⁴ are each CH; or one of X¹, X², X³ and X⁴ is N and the others are CH;

R¹ is OCF₃ or CF₃;

R² is C₁₋₆ alkyl;

R³ is NR(CH₂)₂NR⁴R⁵ or a 5-7 membered heterocyclic ring; or R³ is aryl or a 5-7 membered heteroaryl, each of which is optionally substituted with 1-2 R⁶ groups or optionally substituted with an aryl or heteroaryl, each of which is optionally substituted with 1-2 R^{6a} groups; wherein R⁶ and R^{6a} are independently CONR(CH₂)ₙOR⁷, CONR(CH₂)ₙNR⁴R⁵, CONR⁴R⁵, NR(CH₂)ₙOR⁷, NR(CH₂)ₙNR⁴R⁵, SO₂NRR⁷, NR⁴R⁵ or SO₂R⁸;

R⁴ is H or C₁₋₆ alkyl;

R⁵ is H, C₁₋₆ alkyl, aryl or heteroaryl;

alternatively, R⁴ and R⁵ together with N in NR⁴R⁵ may form a 5-7 membered ring;

R and R⁷ are independently H or C₁₋₆ alkyl;

R⁸ is C₁₋₆ alkyl;

m is 0-1; and

n is 1-4;

provided said ATP-competitive inhibitor is not imatinib when said non-ATP competitive inhibitor is

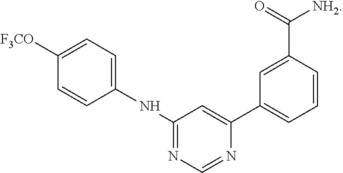

In one embodiment, the non-ATP competitive inhibitor binds to the myristate binding site of BCR-ABL. In some embodiments, the non-ATP competitive inhibitor is a compound of Formula (2):

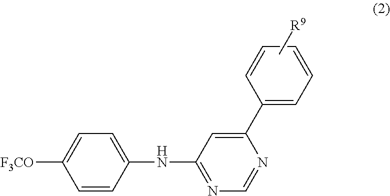

wherein R⁹ is in the meta or para-position, and is selected from carboxamido, CONH(CH₂)₂OH, sulfones (SO₂CH₃) or sulfonamides (SO₂NHR).

In some examples, X¹, X², X³ and X⁴ in Formula (1) are each CH. In other examples, R¹ in Formula (1) is OCF₃

In other embodiments, R³ in Formula (1) is morpholinyl, imidazolyl or pyridyl, wherein said pyridyl is optionally substituted with 1 R^{6a} group; and R^{6a} is as defined in Formula (1). In other examples, R³ is phenyl and is optionally substituted in the meta- or para-position with 1 R⁶ group as defined in Formula (1). In yet other examples, R³ in Formula (1) is NR(CH₂)₂NR⁴R⁵, and R⁴ and R⁵ together with N form morpholinyl.

In particular embodiments, the invention provides a composition comprising an ATP-competitive BCR-ABL inhibitor selected from imatinib, nilotinib and dasatinib; and a non-ATP competitive BCR-ABL inhibitor selected from

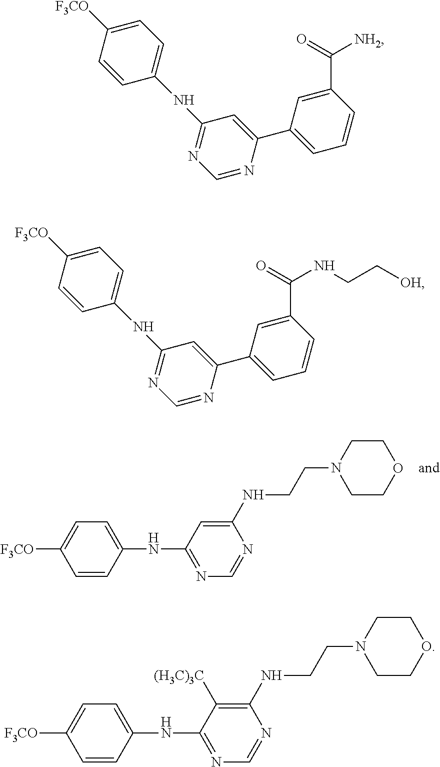

In other embodiments, the invention provides a composition comprising nilotinib and a non-ATP competitive BCR-ABL inhibitor selected from

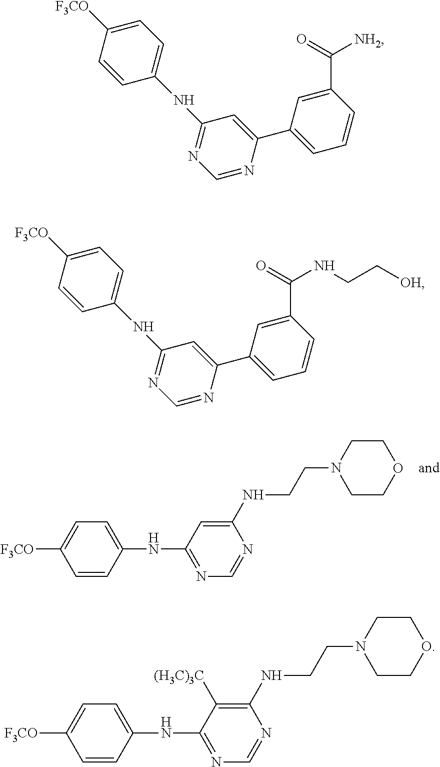

In another aspect, the invention provides methods for treating cancers, particularly a BCR-ABL positive leukemia, comprising administering to a system or a subject, a therapeutically effective amount of a composition comprising an ATP-competitive BCR-ABL inhibitor and a non-ATP competitive BCR-ABL inhibitor as described above, thereby treating said BCR-ABL positive leukemia. For example, the compositions of the invention may be used to treat chronic myeloid leukemia or acute lymphocyte leukemia.

Furthermore, the present invention provides for the use of a therapeutically effective amount of a composition comprising an ATP-competitive BCR-ABL inhibitor and a non-ATP competitive BCR-ABL inhibitor as described above, in the manufacture of a medicament for treating a cell proliferative disorder, particularly BCR-ABL positive leukemia.

In the above compositions and methods for using the compositions of the invention, the inventive composition may be administered to a system comprising cells or tissues. In some embodiments, the invention composition may be administered to a human or animal subject.

### BRIEF DESCRIPTION OF THE DRAWINGS

FIG. 1 shows the effect of various concentrations of GNF-2, imatinib or combinations of both on the number of emerging Ba/F3.BCR-ABL resistant clones.

FIG. 2 shows GNF-5 plasma concentration (nanomolar) versus time (hours) following intravenous and oral doses of 5 mg/kg and 20 mg/kg respectively.

FIG. 3A shows quantification of tumor/control for wild-type luciferase expressing Ba/F3.p210 cells on days five and seven after treatment with vehicle, GNF-5 50 mg/kg and 100 mg/kg b.i.d. FIG. 3B shows the effects of GNF-5, nilotinib and varying concentrations of GNF-5 in combination with nilotinib (0.3-10 μM) on the proliferation of T315I BCR-ABL expressing Ba/F3 cells. FIG. 3C shows the effects of varying concentrations of GNF-5 in combination with nilotinib (0.6-20 μM) on the proliferation of T315I BCR-ABL and T315I/E505K BCR-ABL expressing Ba/F3 cells.

FIG. 4A shows average white blood cell counts for vehicle and 50 mg/kg b.i.d. nilotinib or 75 mg/kg b.i.d. GNF-5 or combination (nilotinib 50 mg/kg b.i.d.+GNF-5 75 mg/kg b.i.d.) treatments in T315I BCR-ABL bone marrow transplantation efficacy study. FIG. 4B shows spleen weight for vehicle and 50 mg/kg b.i.d. nilotinib or 75 mg/kg b.i.d. GNF-5 or combination (nilotinib 50 mg/kg b.i.d.+GNF-5 75 mg/kg b.i.d.) treatments in T315I BCR-ABL bone marrow transplantation efficacy study. FIG. 4C shows time course inhibition of Stat5 phosphorylation after a single dose of GNF-5 and nilotinib combination. FIG. 4D shows a Kaplan-Meier plot showing survival of mice (n=5 mice per group) transplanted with T315I BCR-ABL transduced bone marrow and treated with vehicle (solid line), 75 mg/kg b.i.d.GNF-5 (dotted line), 50 mg/kg b.i.d. nilotinib (dots and dashes), or a combination of 75 mg/kg b.i.d. GNF-5 plus 50 mg/kg b.i.d. nilotinib (dashed line). Compound dosing was initiated on day 11 post-transplantation and discontinued on day 50 (indicated by arrows).

### DEFINITIONS

Unless defined otherwise, all technical and scientific terms used herein have the same meaning as commonly understood by those of ordinary skill in the art to which this invention pertains. The following references provide one of skill with a general definition of many terms used in this invention: Oxford Dictionary of Biochemistry and Molecular Biology, Smith et al. (eds.), Oxford University Press (revised ed., 2000); Dictionary of Microbiology and Molecular Biology, Singleton et al. (eds.), John Wiley & Sons (3^{rd} ed., 2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, one skilled in the art would expect an analog to exhibit the same, similar, or improved utility. Synthesis and screening of analogs to identify variants of known compounds having improved traits (such as higher binding affinity for a target molecule) is an approach that is well known in pharmaceutical chemistry.

As used herein, “contacting” has its normal meaning and refers to combining two or more molecules (e.g., a small molecule organic compound and a polypeptide) or combining molecules and cells (e.g., a compound and a cell). Contacting can occur in vitro, e.g., combining two or more agents or combining a compound and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

The term “inhibiting” or “inhibition,” in the context of tumor growth or tumor cell growth, refers to delayed appearance of primary or secondary tumors, slowed development of primary or secondary tumors, decreased occurrence of primary or secondary tumors, slowed or decreased severity of secondary effects of disease, or arrested tumor growth and regression of tumors. The term “prevent” or “prevention” refers to a complete inhibition of development of primary or secondary tumors or any secondary effects of disease. In the context of modulation of enzymatic activities, inhibition relates to reversible suppression or reduction of an enzymatic activity including competitive, uncompetitive, and noncompetitive inhibition. This can be experimentally distinguished by the effects of the inhibitor on the reaction kinetics of the enzyme, which may be analyzed in terms of the basic Michaelis-Menten rate equation. Competitive inhibition occurs when the inhibitor can combine with the free enzyme in such a way that it competes with the normal substrate for binding at the active site. A competitive inhibitor reacts reversibly with the enzyme to form an enzyme-inhibitor complex [EI], analogous to the enzyme-substrate complex.

The term “modulate” with respect to a biological activity of a reference protein or its fragment refers to a change in the expression level or other biological activities of the protein. For example, modulation may cause an increase or a decrease in expression level of the reference protein, enzymatic modification (e.g., phosphorylation) of the protein, binding characteristics (e.g., binding to another molecule), or any other biological (e.g., enzymatic), functional, or immunological properties of the reference protein. The change in activity can arise from, for example, an increase or decrease in expression of one or more genes that encode the reference protein, the stability of an mRNA that encodes the protein, translation efficiency, or from a change in other biological activities of the reference protein. The change can also be due to the activity of another molecule that modulates the reference protein (e.g., a kinase which phosphorylates the reference protein). Modulation of a reference protein can be up-regulation (i.e., activation or stimulation) or down-regulation (i.e. inhibition or suppression). The mode of action of a modulator of the reference protein can be direct, e.g., through binding to the protein or to genes encoding the protein, or indirect, e.g., through binding to and/or modifying (e.g., enzymatically) another molecule which otherwise modulates the reference protein.

The term “subject” includes mammals, especially humans. It also encompasses other non-human animals such as cows, horses, sheep, pigs, cats, dogs, mice, rats, rabbits, guinea pigs, monkeys.

The term “treat” or “treatment” refers to arrested tumor growth, and to partial or complete regression of tumors. The term “treating” includes the administration of compounds or agents to prevent or delay the onset of the symptoms, complications, or biochemical indicia of a disease (e.g., leukemia), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

### MODES OF CARRYING OUT THE INVENTION

The invention provides compositions thereof, which may be useful for inhibiting tumor cell growth and for treating a variety of cancers.

In one aspect, the invention provides a composition comprising an ATP-competitive BCR-ABL inhibitor and a non-ATP competitive BCR-ABL inhibitor;

wherein the ATP-competitive inhibitor is selected from the group consisting of imatinib (STI571), nilotinib (AMN107), pyrido[2,3-d]pyrimidine compounds (e.g., dasatinib), bosutinib, 3-substituted benzamide derivatives (e.g., INNO-406), AZD-0530, MK-0457, PHA-739358, AP24534 (Ariad), JNJ-26483327(Johnson & Johnson), HPK-61 (SuperGen), SKS-927 (Wyeth), AT-9283 (Astex Pharmaceuticals), EXEL-2280 (Exelisis) and TG-100572 (Targegen);

wherein said ATP non-competitive BCR-ABL inhibitor is a compound of Formula (1):

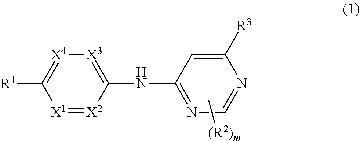

or a pharmaceutically acceptable salt thereof;

wherein X¹, X², X³ and X⁴ are each CH; or one of X¹, X², X³ and X⁴ is N and the others are CH;

R¹ is OCF₃ or CF₃;

R² is C₁₋₆ alkyl;

R³ is NR(CH₂)₂NR⁴R⁵ or a 5-7 membered heterocyclic ring; or R³ is aryl or a 5-7 membered heteroaryl, each of which is optionally substituted with 1-2 R⁶ groups or optionally substituted with an aryl or heteroaryl, each of which is optionally substituted with 1-2 R^{6a} groups; wherein R⁶ and R^{6a} are independently CONR(CH₂)ₙOR⁷, CONR(CH₂)ₙNR⁴R⁵, CONR⁴R⁵, NR(CH₂)ₙOR⁷, NR(CH₂)ₙNR⁴R⁵, SO₂NRR⁷, NR⁴R⁵ or SO₂R⁸;

R⁴ is H or C₁₋₆ alkyl;

R⁵ is H, C₁₋₆ alkyl, aryl or heteroaryl;

alternatively, R⁴ and R⁵ together with N in NR⁴R⁵ may form a 5-7 membered ring;

R and R⁷ are independently H or C₁₋₆ alkyl;

R⁸ is C₁₋₆ alkyl;

m is 0-1; and

n is 1-4;

provided said ATP-competitive inhibitor is not imatinib when said non-ATP competitive inhibitor is

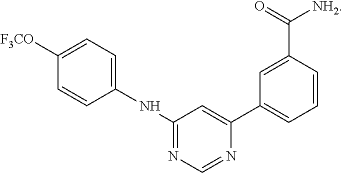

The invention also provides methods for treating cancers, particularly a BCR-ABL positive leukemia, comprising administering to a system or a subject, a therapeutically effective amount of a composition comprising an ATP-competitive BCR-ABL inhibitor and a non-ATP competitive BCR-ABL inhibitor as described above, thereby treating said BCR-ABL positive leukemia. For example, the compositions of the invention may be used to treat chronic myeloid leukemia or acute lymphocyte leukemia.

Chronic Myelogenous Leukemia (CML) is a hematological disorder caused by a chromosomal rearrangement that generates a fusion protein, BCR-ABL, with deregulated tyrosine kinase activity. Imatinib, a small-molecule ABL kinase inhibitor and a highly effective therapy for early-phase chronic myeloid leukemia (CML), has constitutively active ABL kinase activity owing to the expression of the BCR-ABL fusion protein. However, there is a high relapse rate among advanced- and blast-crisis-phase patients owing to the development of mutations in the ABL kinase domain that cause drug resistance.

Mutations that cause imatinib resistance are usually those that lead to a BCR-ABL protein with a functional ABL tyrosine kinase domain, but that abrogate or impair drug binding. Point mutations in BCR-ABL reduce the binding of imatinib to the protein by either a direct or an indirect mechanism. In the case of direct mechanisms, mutations are clustered around the imatinib binding site, which partially overlaps that of ATP, and reduce imatinib binding either as a result of changes to amino-acid side-chains, or as a result of topographical changes that sterically hinder imatinib binding. Examples of residues that inhibit imatinib binding when they are mutated are Thr315 and Phe317. (Weisberg et al., Nat. Rev. Cancer 7:345-56 (2007)).

Mutations that inhibit imatinib binding through an indirect mechanism exploit the particular binding mode of the drug to its target protein Imatinib binds to a catalytically inactive conformation of the ABL kinase domain, often referred to as the ‘DFG-out’ conformation, in which the highly conserved Asp-Phe-Gly (DFG) triad is flipped out of its usual position in active kinase conformations. This makes a channel beyond the Thr315 gatekeeper residue that opens up an auxiliary binding site, which is occupied by the piperazinyl-substituted benzamide moiety of imatinib. (Weisberg et al., Nat. Rev. Cancer (2007), supra).

Although clinical remission of CML has been achieved with the ATP-site targeting drug imatinib, many patients relapse due to emergence of clones expressing inhibitor-resistant forms of BCR-ABL. One strategy to overcome resistance mutations is to design new ATP-competitive inhibitors that derive potency and selectivity from alternative binding modes. This approach has been clinically validated by the development of dasatinib and nilotinib that are capable of circumventing all known mutations with the exception of T315I. Although T315I BCR-ABL targeting compounds have been developed, it is exceedingly difficult for them to retain a moderate level of selectivity because the gatekeeper position is one of the most important selectivity determinants for kinase inhibitors. For example, two potent ATP-site directed agents have been advanced to clinical testing: nilotinib (AMN107) and dasatinib (BMS-354825). Although both compounds inhibit most of the mutations that induce resistance to imatinib, neither compound is capable of inhibiting the “gatekeeper” T315I mutation, which is situated in the middle of the ATP-binding cleft. (Gone et al., Science 293:876-880 (2001); O'Hare et al., Cancer Res. 65:4500-4505 (2005)).

Another strategy is to find non-ATP competitive inhibitors that utilize binding sites that are able to allosterically regulate kinase activity. Although they are not as easily discovered and characterized as ATP-competitive inhibitors, allosteric inhibitors have been found for kinases such as mTor¹⁹, Mek²⁰ Akt²¹, IKK²² and CAMK. A major advantage of non-ATP competitive kinase inhibitors is that they can be highly selective for a particular kinase because they can exploit non-conserved kinase regulatory mechanisms. GNF-2 (Adrian et al., Nature Chem. Biol. 2:95-102 (2006)) demonstrated exclusive cellular activity against BCR-ABL transformed cells (IC₅₀=140 nM), and did not inhibit the activity of 40 other tyrosine kinases in cellular assays or the biochemical activity of a panel of 80 diverse kinases. Furthermore, because GNF-2 occupies an independent binding site, there is the potential for it to act synergistically with ATP-competitive compounds.

GNF-2 has been shown to target cellular BCR-ABL by purifying BCR-ABL from cellular extracts by affinity chromatography with the immobilized inhibitor, by demonstrating inhibition of cellular BCR-ABL autophosphorylation and of downstream Stat5 phosphorylation, and by the ability of mutations located in the ATP-site (T315I) or in the myristate binding site (A337N and A344L) of BCR-ABL to induce resistance to the compound. As a first step towards further elucidating the molecular mechanism by which GNF-2 inhibits BCR-ABL dependent cell growth, we have established and characterized the binding site of GNF-2 to Abl using NMR. Simultaneous binding of a myristoyl mimic and an ATP-competitive inhibitor to BCR-ABL reduces the appearance of resistance-conferring mutations and results in inhibition of wild type and T315I mutant BCR-ABL driven cell growth in vivo.

Although the conformational rearrangement and/or recruitment of additional cellular co-factors upon GNF-2 binding to BCR-ABL remains to be elucidated, GNF-2 appears to be able to exploit a regulatory mechanism that is normally functional with c-Abl but that is lost in BCR-ABL due to fusion of the Bcr-domain. As discussed below, ATP competitive and myristate-targeting inhibitors can bind to BCR-ABL simultaneously and appear to cooperate in stabilizing the “closed” inactive conformation of the kinase. While non-ATP competitive inhibitors will also be subjected to inhibitor resistance through point mutation, the combined application of ATP and non-ATP competitive inhibitors reduces the number of resistant clones that emerge as a response to continued exposure to a single agent. Furthermore, the combined treatment of GNF-5 with nilotinib led to in vivo efficacy resulting in complete disease remissions in a T315I BCR-ABL mutant murine bone-marrow transplantation model.

A. Non-ATP Competitive BCR-ABL Inhibitors

Various non-ATP competitive BCR-ABL inhibitors that are known in the art to inhibit BCR-ABL by targeting sites remote from the ATP binding site may be used to practice the invention. In particular embodiments, the non-ATP competitive BCR-ABL inhibitors for use in the present invention bind to the myristate binding site of BCR-ABL. Examples of non-ATP competitive BCR-ABL inhibitors for use in the present invention include but not limited to compounds described in WO 04/089286, which is incorporated herein by reference in its entirety; and compounds having Formula (1):

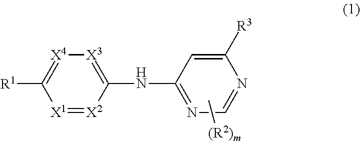

or a pharmaceutically acceptable salt thereof;

wherein X¹, X², X³ and X⁴ are each CH; or one of X¹, X², X³ and X⁴ is N and the others are CH;

R¹ is OCF₃ or CF₃;

R² is C₁₋₆ alkyl;

R³ is NR(CH₂)₂NR⁴R⁵ or a 5-7 membered heterocyclic ring; or R³ is aryl or a 5-7 membered heteroaryl, each of which is optionally substituted with 1-2 R⁶ groups or optionally substituted with an aryl or heteroaryl, each of which is optionally substituted with 1-2 R^{6a} groups; wherein R⁶ and R^{6a} are independently CONR(CH₂)ₙOR⁷, CONR(CH₂)ₙNR⁴R⁵, CONR⁴R⁵, NR(CH₂)ₙOR⁷, NR(CH₂)ₙNR⁴R⁵, SO₂NRR⁷, NR⁴R⁵ or SO₂R⁸;

R⁴ is H or C₁₋₆ alkyl;

R⁵ is H, C₁₋₆ alkyl, aryl or heteroaryl;

alternatively, R⁴ and R⁵ together with N in NR⁴R⁵ may form a 5-7 membered ring;

R and R⁷ are independently H or C₁₋₆ alkyl;

R⁸ is C₁₋₆ alkyl;

m is 0-1; and

n is 1-4.

Table 1 shows examples of compounds having Formula (1) which may be used as non-ATP competitive BCR-ABL inhibitors.

TABLE 1

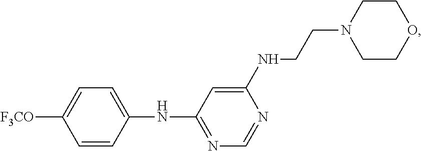

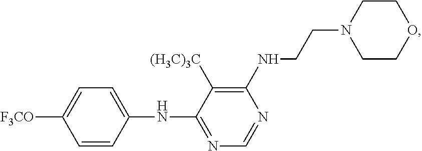

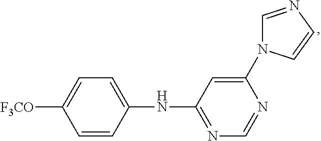

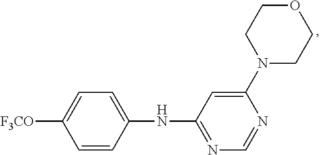

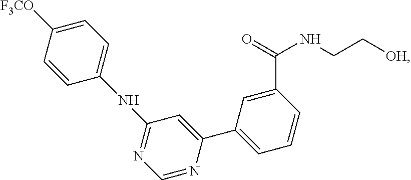

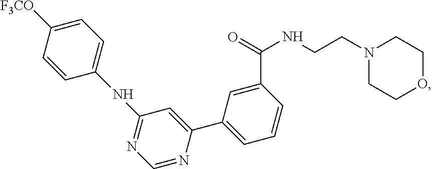

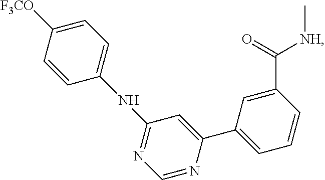

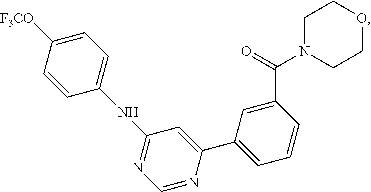

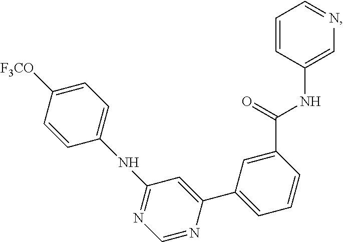

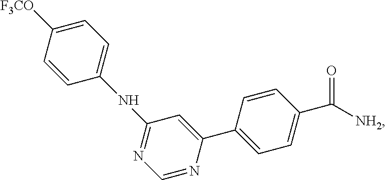

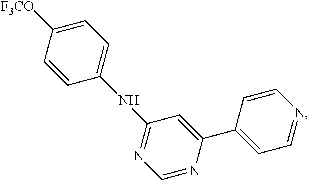

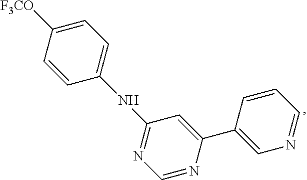

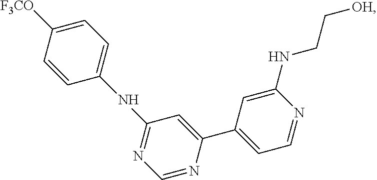

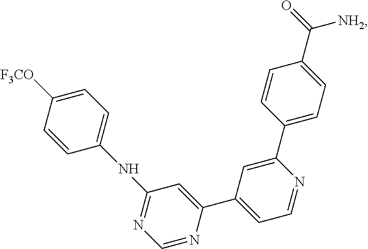

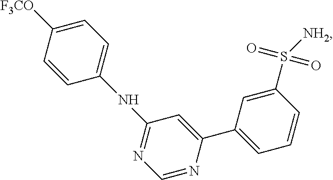

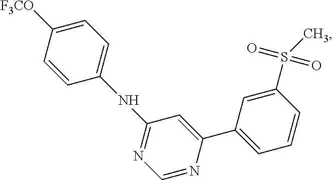

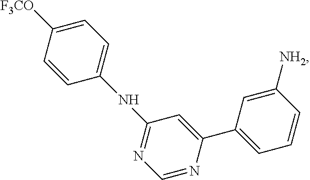

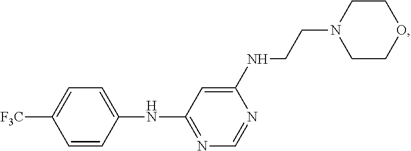

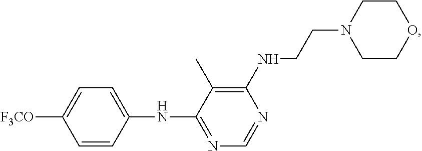

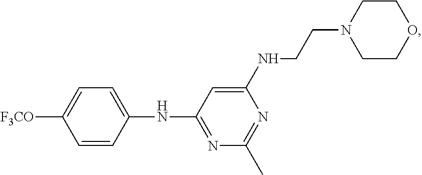

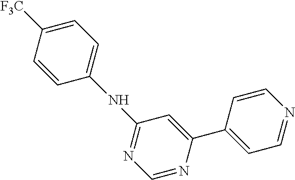

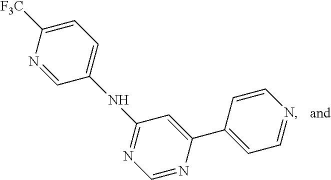

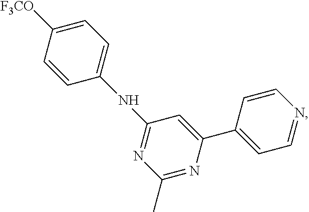

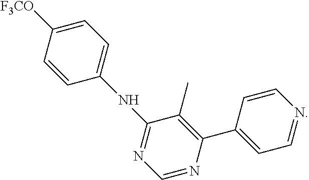

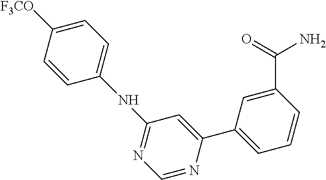

B. ATP Competitive BCR-ABL Inhibitors

Various ATP-competitive BCR-ABL inhibitors that are known in the art to inhibit BCR-ABL by targeting the ATP binding site may be used to practice the invention, including but not limited to ABL inhibitors, inhibitors of both ABL and Src-family kinases, and Aurora kinase inhibitors.

The Src family of tyrosine kinases modulates multiple intracellular signal transduction pathways involved in cell growth, differentiation, migration and survival, many of which are involved in oncogenesis, tumor metastasis and angiogenesis. (Weisberg et al., Nat. Rev. Cancer 7:345-356 (2007)). Many kinases from the Src family are expressed in hematopoietic cells (Blk, Fgr, Fyn, Hck, Lck, Lyn, c-Src and Yes). In addition, BCR-ABL has been shown to be capable of activating Src kinases both through phosphorylation and merely by binding Src proteins. Furthermore, cell lysates from imatinib-resistant patients have been found to over-express Lyn kinase, and the proliferation of human CML K562 cells selected for resistance to imatinib, which also over-express Lyn, is inhibited by the Abl/Src inhibitor, PD180970. Since Src family kinases regulate downstream elements of the BCR-ABL signaling cascade, inhibition of these enzymes could therefore provide synergy with BCR-ABL inhibition, and potentially counteract the availability of alternative survival pathways which CML cells could utilize in the face of BCR-ABL inhibition. Therapy with combined BCR-ABL and Src-family kinase inhibitors might also therefore counteract the oncogenic potential of drug-resistant mutant forms of BCR-ABL in CML and/or ALL. (Manley et al., Biochim Biophys. Acta 1754:3-13 (2005)). Dasatinib (BMS-354825), bosutinib (SKI-606), INNO-404 (NS-187) and AZD05030 are examples of dual ABL-Src inhibitors.

The Aurora family of serine/threonine kinases is important for mitotic progression. Aurora-A has been reported to be overexpressed in various human cancers, and its overexpression induces aneuploidy, centrosome amplification and tumorigenic transformation in cultured human and rodent cells. (Zhang et al., Oncogene 2004, 23:8720-30). MK-0457 (Merck; originally developed by Vertex Pharmaceuticals as VX-680), a potent inhibitor of all three Aurora kinases and FLT3 in the nanomolar range, is a moderate to strong inhibitor of ABL and JAK2, which are relevant targets for a range of myeloproliferative disorders. MK-0457 also inhibits the autophosphorylation of T315I mutant BCR-ABL in transformed Ba/F3 cells with an IC₅₀ of ˜5 μM, although it inhibits cell proliferation at submicromolar concentrations.

Table 2 shows exemplary ATP-competitive BCR-ABL inhibitors which may be used to practice the invention, including imatinib (STI571), nilotinib (AMN107), pyrido[2,3-d]pyrimidine compounds (e.g., dasatinib), bosutinib, 3-substituted benzamide derivatives (e.g., INNO-406), AZD-0530, MK-0457, PHA-739358, AP24534 (Ariad), JNJ-26483327(Johnson & Johnson), HPK-61 (SuperGen), SKS-927 (Wyeth), AT-9283 (Astex Pharmaceuticals), EXEL-2280 (Exelisis) and TG-100572 (Targegen). (See e.g., Weisberg et al., Nat. Rev. Cancer (2007), supra; Das et al., J. Med. Chem. 49:6819-6832 (2006); Puttini et al., Cancer Res. 66:11314-11322 (2006); Kimura et al., Blood 106:3948-3954 (2005); Hennequin et al., J. Med. Chem. 49:6465-6488 (2006); each of which is hereby incorporated by reference).

TABLE 2

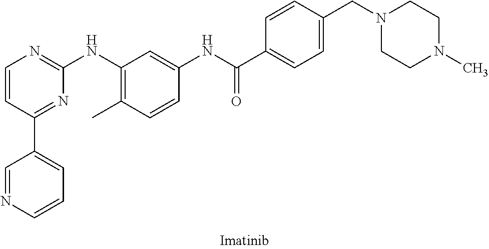

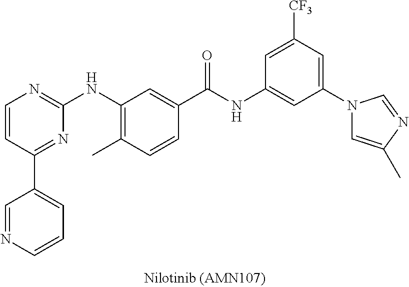

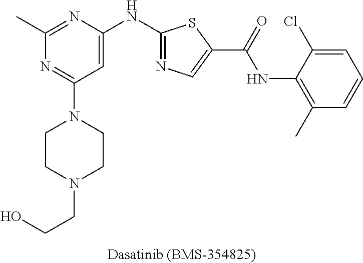

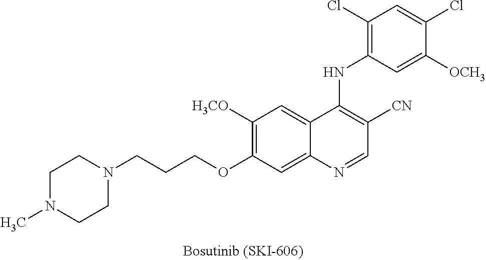

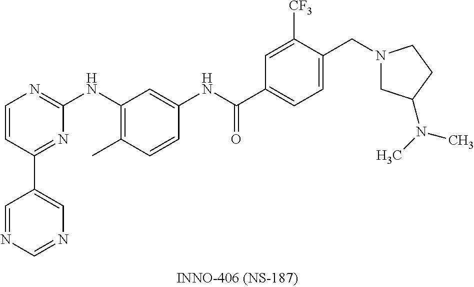

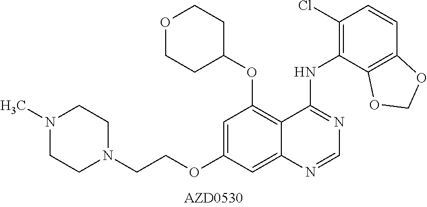

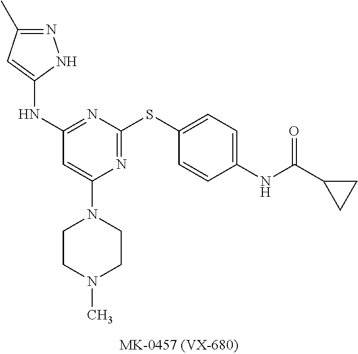

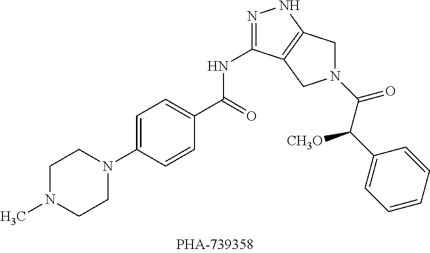

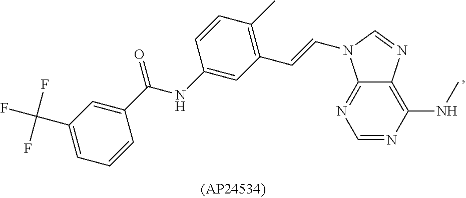

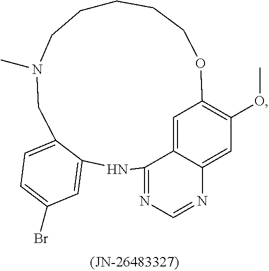

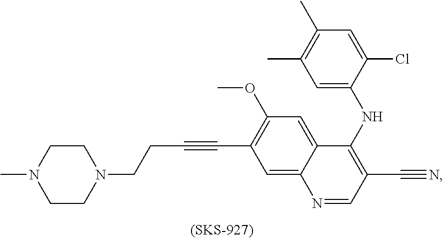

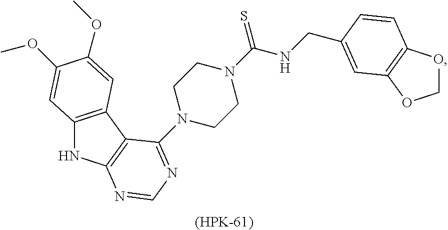

AT-9283 (Astex Therapeutics), EXEL-2280 (Exelisis), and TG-100572 (TargeGen).

C. Diseases and Conditions to be Treated

The combination of the present invention may be used for treating a variety of cancers. In one embodiment, the invention provides an ATP-competitive BCR-ABL inhibitor in combination with a non-ATP competitive BCR-ABL inhibitor, for inhibiting the growth and proliferation of hematopoietic tumors of lymphoid lineage including leukemia, acute lymphocytic leukemia (ALL), acute lymphoblastic leukemia, B-cell lymphoma, T-cell lymphoma, Hodgkins lymphoma, non-Hodgkins lymphoma, hairy cell lymphoma, histiocytic lymphoma, and Burkitts lymphoma; and hematopoietic tumors of myeloid lineage including acute and chronic myelogenous leukemias (CML), myelodysplastic syndrome, myeloid leukemia, and promyelocytic leukemia. The combination of the present invention is also useful for treating cancers known to be associated with BCR-ABL. In particular embodiments, the combination of the present invention may be used for treating BCR-ABL-positive CML and ALL.

Chronic myelogenous leukemia (CML) is a cancer of the bone marrow characterized by increased and unregulated clonal proliferation of predominantly myeloid cells in the bone marrow. Its annual incidence is 1-2 per 100,000 people, affecting slightly more men than women. CML represents about 15-20% of all cases of adult leukemia in Western populations, about 4,500 new cases per year in the U.S. or in Europe. (Faderl et al., N. Engl. J. Med. 341: 164-72 (1999)).

CML is a clonal disease that originates from a single transformed hematopoietic stem cell (HSC) or multipotent progenitor cell (MPP) harboring the Philadelphia translocation t(9/22). The expression of the gene product of this translocation, the fusion oncogene BCR-ABL, induces molecular changes which result in expansion of the malignant hematopoiesis including the leukemic stem cell (LSC) pool and the outgrowth and suppression of non-malignant hematopoiesis (Stam et al., Mol Cell Biol. 7:1955-60 (1987)). During the course of the disease, the leukemic stem cell pool expands and in the final stage, the blast crisis, nearly all CD34+CD38-cells carry the Philadelphia translocation.

Imatinib mesylate (STI571, GLEEVEC®) is becoming the standard of therapy for CML with response rates of more than 96%, and works by inhibiting the activity of BCR-ABL. However, despite initial success, patients eventually develop resistance to imatinib mesylate due to acquisition of point mutations in BCR-ABL. In view of the limitations of imatinib mesylate, there is a need for improved methods for treating CML.

In addition, it is contemplated that the combination of the present invention may be used for treating carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); tumors of the central and peripheral nervous system including astrocytoma, neuroblastoma, glioma, and schwannomas; tumors of mesenchymal origin including fibrosarcoma, rhabdomyosarcoma, and osteosarcoma; and other tumors including melanoma, xeroderma pigmentosum, keratoacanthoma, seminoma, thyroid follicular cancer, and teratocarcinoma. It is also contemplated that the combinations of the present invention may be used for treating mastocytosis, germ cell tumors, pediatric sarcomas, and other cancers.

The therapeutic methods described herein may be used in combination with other cancer therapies. For example, Hh antagonists in combination with BCR-ABL inhibitors may be administered adjunctively with any of the treatment modalities, such as chemotherapy, radiation, and/or surgery. For example, they can be used in combination with one or more chemotherapeutic or immunotherapeutic agents; and may be used after other regimen(s) of treatment is concluded. Examples of chemotherapeutic agents which may be used in the compositions and methods of the invention include but are not limited to anthracyclines, alkylating agents (e.g., mitomycin C), alkyl sulfonates, aziridines, ethylenimines, methylmelamines, nitrogen mustards, nitrosoureas, antibiotics, antimetabolites, folic acid analogs (e.g., dihydrofolate reductase inhibitors such as methotrexate), purine analogs, pyrimidine analogs, enzymes, podophyllotoxins, platinum-containing agents, interferons, and interleukins.

Particular examples of known chemotherapeutic agents which may be used in the compositions and methods of the invention include, but are not limited to, busulfan, improsulfan, piposulfan, benzodepa, carboquone, meturedepa, uredepa, altretamine, triethylenemelamine, triethylenephosphoramide, triethylenethiophosphoramide, trimethylolomelamine, chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, doxorubicin, epirubicin, mitomycin C, mycophenolic acid, nogalamycin, olivomycin, peplomycin, plicamycin, porfiromycin, puromycin, streptonigrin, streptozocin, tubercidin, ubenimex, zinostatin, zorubicin, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, fluorouracil, tegafur, L-asparaginase, pulmozyme, aceglatone, aldophosphamide glycoside, aminolevulinic acid, amsacrine, bestrabucil, bisantrene, carboplatin, cisplatin, defofamide, demecolcine, diaziquone, elformithine, elliptinium acetate, etoglucid, etoposide, flutamide, gallium nitrate, hydroxyurea, interferon-alpha, interferon-beta, interferon-gamma, interleukin-2, lentinan, lonidamine, prednisone, dexamethasone, leucovorin, mitoguazone, mitoxantrone, mopidamol, nitracrine, pentostatin, phenamet, pirarubicin, podophyllinic acid, 2-ethylhydrazide, procarbazine, razoxane, sizofuran, spirogermanium, paclitaxel, tamoxifen, teniposide, tenuazonic acid, triaziquone, 2,2′,2″-trichlorotriethylamine, urethane, vinblastine, vincristine, and vindesine.

The present methods may be used to treat primary, relapsed, transformed, or refractory forms of cancer. Often, patients with relapsed cancers have undergone one or more treatments including chemotherapy, radiation therapy, bone marrow transplants, hormone therapy, surgery, and the like. Of the patients who respond to such treatments, they may exhibit stable disease, a partial response (i.e., the tumor or a cancer marker level diminishes by at least 50%), or a complete response (i.e., the tumor as well as markers become undetectable). In either of these scenarios, the cancer may subsequently reappear, signifying a relapse of the cancer.

D. Pharmaceutical Compositions and Administration

The compositions of the present invention may be administered alone under sterile conditions to a subject in need of treatment. In particular embodiments, they are administered as an active ingredient of a pharmaceutical composition. Pharmaceutical compositions of the present invention may comprise an effective amount of an agent that inhibits the hedgehog signaling pathway in combination with an agent that inhibits BCR-ABL, together with one or more acceptable carriers thereof. The compositions may also contain a third therapeutic agent noted above, e.g., a chemotherapeutic agent or other anti-cancer agent.

Pharmaceutical carriers enhance or stabilize the composition, or facilitate preparation of the composition. Pharmaceutically acceptable carriers are determined in part by the particular composition being administered (e.g., nucleic acid, protein, or other type of compounds), as well as by the particular method used to administer the composition. They should also be both pharmaceutically and physiologically acceptable in the sense of being compatible with the other ingredients and not injurious to the subject. They may take a wide variety of forms depending on the form of preparation desired for administration, e.g., oral, sublingual, rectal, nasal, or parenteral. For example, an antitumor compound may be complexed with carrier proteins such as ovalbumin or serum albumin prior to their administration in order to enhance stability or pharmacological properties.

There are a wide variety of suitable formulations of pharmaceutical compositions of the present invention (see, e.g., Remington: The Science and Practice of Pharmacy, Mack Publishing Co., 20^{th} ed., 2000). Without limitation, pharmaceutically acceptable carriers include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The carrier may also include a sustained release material such as glyceryl monostearate or glyceryl distearate, alone or with a wax.

The pharmaceutical compositions may be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

The therapeutic formulations may be delivered by any effective means that may be used for treatment. Depending on the specific antitumor agent to be administered, the suitable means include oral, nasal, pulmonary administration, or parenteral (including subcutaneous, intramuscular, intravenous and intradermal) infusion into the bloodstream. For parenteral administration, antitumor agents of the present invention may be formulated in a variety of ways. Aqueous solutions of the modulators may be encapsulated in polymeric beads, liposomes, nanoparticles or other injectable depot formulations known to those of skill in the art. Additionally, the compounds of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The therapeutic formulations may conveniently be presented in unit dosage form and administered in a suitable therapeutic dose. A suitable therapeutic dose may be determined by any well known methods such as clinical studies on mammalian species to determine maximum tolerable dose and on normal human subjects to determine safe dosage. Except under certain circumstances when higher dosages may be required, the dosage of an antitumor agent of the present invention usually lies within the range of from about 0.001 to about 1000 mg, more usually from about 0.01 to about 500 mg per day. The dosage and mode of administration of an antitumor agent may vary for different subjects, depending upon factors that may be individually reviewed by the treating physician, such as the condition or conditions to be treated, the choice of composition to be administered, including the particular antitumor agent, the age, weight, and response of the individual subject, the severity of the subject's symptoms, and the chosen route of administration. As a general rule, the quantity of an antitumor agent administered is the smallest dosage which effectively and reliably prevents or minimizes the conditions of the subjects. Therefore, the above dosage ranges are intended to provide general guidance and support for the teachings herein, but are not intended to limit the scope of the invention.

### EXAMPLES

The following examples are provided to illustrate, but not to limit the present invention. All animal experiments are in accordance with the US National Institutes of Health Statement of Compliance with Standards for Humane Care and Use of Laboratory Animals. All NMR experiments are carried out at 296 K on a Bruker AV600 NMR spectrometer at a proton resonance frequency of 600 MHz as described in Strauss et al., J. Biomol. NMR 31:343-349 (2005).

### Example 1

### General Materials and Methods

Abl Crystallography

Crystals were grown as described in Nagar et al. (Cancer Res 62, 4236-43 (2002)) using the conditions listed in Table 3A. After soaking for 7 days at 4° C. in an excess of GNF-2, data were collected from a single crystal at beamline PXII of the Swiss Light Source.

TABLE 3A

| Protein solution | 20 mg/ml |
|---|---|
| Crystallization buffer | 0.1M MES pH 5.6, |
| | 0.2M MgCl₂, |
| | 18% PEG4000 |
| Wavelength (Å) | 1.00160 |
| Space group | P1 |
| Number of molecules in A.U. | 2 |
| Unit Cell (Å; degrees) | 42.1, 65.3, 66.3; 72.8, 80.2, 84.9 |
| Resolution range (highest shell) (Å) | 62.50-1.74 (1.80-1.74) |
| Rsym (%) | 4.5 (38.2) |
| I/sig(I) (%) | 16.5 (3.4) |
| Completeness (%) | 95.3 (86.2) |
| Multiplicity (%) | 3.9 (3.6) |
| Observed reflections (Unique) | 254609 (64929) |
| Wilson B-factor (Å²) | 31.8 |

Statistics of the refinement and details of ligand-protein interactions in the final model are listed in Tables 3B and 3C. Table 3C shows distances between protein and ligand less than or equal to 3.8 Å. Distances greater than 3.8 Å are not listed.

TABLE 3B

| | |
|---|---|
| Resolution range (highest shell) (Å) | 38.95-1.74 (1.79-1.74) |
| Completeness (%) | 95.4 |
| Free R-factor test set (#, %) | 3247, 5.0 |
| R-factor (R-free) | 0.1978 (0.2312) |
| Mean B-factor (Å²) | 32.4 |
| R.m.s.d. bonds (Å) | 0.010 |
| R.m.s.d. angles (degrees) | 1.19 |
| Contents of model (# atoms, | |
| mean B-factors (Å)) | |
| Molecule A (aa's: 243-295, 297-529) | 2337, 25.21 |
| Imatinib A | 37, 19.28 |
| GNF-2 A | 27, 28.99 |
| Molecule B (aa's: 244-293, 297-529) | 2299, 38.62 |
| Imatinib B | 37, 26.92 |
| GNF-2 B | 27, 60.45 |
| Solvent (waters; chloride) | 429, 37.77; 1, 31.53 |
| Ramachandran outliers | Lys264, Arg381 (0.72%) |

TABLE 3C

| Ligand atom | Protein atom | Distance Mol A | Distance Mol B |
|---|---|---|---|
| F1 | Phe 512 CE1 | 3.62 | 3.50 |
| | Leu 360 CA | 3.77 | 3.72 |
| | Leu 360 CD1 | 3.78 | |
| | Ala 363 CB | 3.44 | 3.64 |
| | Leu 448 CD1 | 3.68 | 3.78 |
| F3 | Val 487 CG1 | 3.71 | 3.55 |
| | Leu 448 CD1 | 3.09 | 3.19 |
| F4 | Leu 359 C | 2.94 | 3.00 |
| | Leu 360 N | 3.08 | 3.17 |
| | Leu 360 CA | 3.35 | 3.44 |
| | Leu 359 O | 2.94 | 2.98 |
| | Leu 359 CA | 3.78 | 3.79 |
| | Leu 359 CB | 3.44 | 3.39 |
| | Ile 451 CG2 | 3.50 | 3.49 |
| O5 | Val 487 CG2 | 3.76 | 3.74 |
| | Ile 521 CD1 | 3.60 | 3.76 |
| C6 | Ile 521 CD1 | 3.75 | |
| C7 | Ala 356 O | 3.75 | 3.77 |
| | Leu 359 CB | | 3.66 |
| | Ile 521 CD1 | 3.46 | |
| C9 | Leu 359 CD1 | 3.66 | 3.40 |
| | Ala 356 CB | 3.74 | |
| C12 | Pro 484 CD | 3.68 | 3.70 |
| | Ala 452 CB | 3.63 | 3.66 |
| | Cys 483 CA | 3.80 | |
| N16 | Ala 452 O | | 3.77 |
| N19 | Val 525 CG1 | | 3.77 |
| | Leu 359 CD1 | 3.70 | |
| | Ala 356 CB | 3.66 | |
| C20 | Tyr 454 OH | 3.50 | 3.73 |
| C24 | Glu 481 O | | 3.62 |
| C27 | Leu 529 CD1 | 3.77 | |
| C29 | Leu 529 CD1 | 3.72 | |
| C32 | Glu 481 OE1 | | 3.59 |
| C34 | Glu 481 O | 3.54 | 3.64 |
| O37 | Leu 529 CD1 | 3.16 | 3.65 |
| N16 | 244/6 OW0 | 2.90 | 2.75 |
| 244/6 OW0 | Ala 452 O | 2.88 | 2.87 |
| | Glu 481 O | 2.85 | 2.87 |
| N22 | /44 OW0 | 3.09 | |
| /44 OW0 | Tyr 454 OH | 2.90 | |
| | /136 OW0 | 2.68 | |
| N38 | /136 OW0 | 2.97 | |

Wild-Type and Mutant BCR-ABL Ba/F3 Cellular Proliferation Assays

Viability of wild type and mutant BCR-ABL expressing Ba/F3 cells after a 48 hour treatment with various concentrations of single or combined agents was determined by AlamarBlue® (TREK Diagnostic Systems) reduction method. The combination index (CI) was calculated according to the method of Chou and Talalay¹⁵ using the Calcusyn software.

Selection for Clones Resistant to GNF-2 and Imatinib

Emergence of compound resistant Ba/F3.p210 clones was evaluated as previously described in von Bubnoff, N. et al., Blood 105, 1652-9 (2005). One 96-well plate was used for every compound concentration or combination and the medium was renewed every 3-4 days. The plates were incubated for 21 days and the number of wells with evident cell growth was recorded at days 9 and 21.

Pharmacokinetic Parameters in Mice

Male Balb/c mice were dosed with GNF-5 in PEG400/saline, 1:1 at 5 mg/kg intravenously or 20 mg/kg orally. The compound plasma concentration at any given time point was determined by Liquid Chromatography/Mass Spectrometry (LC/MS/MS). Pharmacokinetic parameters were calculated by non-compartmental regression analysis using Winnonlin 4.0 software (Pharsight, Mountain View, Calif., USA).

In Vivo Efficacy in Ba/F3.p210 Xenograft Model

Female SCID beige mice, 6-8 weeks of age (n=5 for each GNF-5-treated or vehicle control group) were injected via tail vein with 1×10⁶ Ba/F3 cells co-expressing BCR-ABL p210 and luciferase. Three days post-injection, mice were orally dosed twice daily with 50 or 100 mg/kg GNF-5 for seven days. At days 5 and 7, bioluminescence was quantified using luciferin and an IVIS imaging system (Xenogen Corp., Alameda, Calif.).

In Vivo Efficacy in Bone Marrow Transduction/Transplantation Model

Bone marrow cells harvested from 6-8 weeks old 5-FU injected male Balb/c mice were transduced with a pMSCV BCR-ABL wt or T315I BCR-ABL retroviral construct and transplanted into irradiated recipient female Balb/c mice (6-8 weeks). Treatment with GNF-5, nilotinib or vehicle control started on days 7 (wt BCR-ABL) or 15 (T315I) after transplantation for 7 days (10 (wt BCR-ABL) or 4 (T315I BCR-ABL) mice per treatment group). Blood cell counts and spleen size were determined at treatment day 7. Bone marrow cells were isolated, fixed, permeabilized and stained with anti-pStat5 and anti-luciferase antibodies and analyzed by flow cytometry.

For survival studies, the treatment with vehicle, GNF-5, nilotinib, or the combination of both (n=5 mice per group) was initiated 11 days after transplantation and prolonged until day 50 post-transplantation or until the mice had to be sacrificed due to becoming moribund. Overall survival and time to relapse were determined by the Kaplan-Meier method. Statistical significance was assessed using the Kaplan-Meier survival analysis, under the assumption of a normal distribution of normalized ratios with an estimate of variance (α=0.05, two-sided).

### Example 2

### 3-[6-(4-Trifluoromethoxy-phenylamino)-pyrimidin-4-yl]-benzamide (GNF-2)

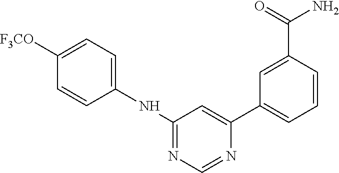

4,6-dichloropyrimidine (1 g, 6.7 mmol) is dissolved with p-trifluoromethoxy aniline (1.2 g, 6.7 mmol) in 15 mL ethanol, then 1.75 mL DIEA (10 mmol) is added. Reaction is under reflux for 2 hours, and cooled down to room temperature. After evaporating the solvent, the crude product is purified by flash chromatography (EA/Hexane=3:7) to give (6-chloro-pyrimidin-4-yl)-(4-trifluoromethoxy-phenyl)-amine as a white solid.

To a degassed solution of (6-chloropyrimidin-4-yl)-(4-trifluoromethoxyphenyl)-amine (73 mg, 0.25 mmol) and (3-aminocarbonylphenyl)-boronic acid (42 mg, 0.25 mmol) in 0.4 M sodium carbonate aqueous solution (1.3 mL) and acetonitrile (1.3 mL) is added PPh₃ (15 mg, 0.01 mmol). After stirring at about 90° C. under N₂ for 12 hours, the reaction mixture is partitioned between saturated NaHCO₃ and CHCl₃/2-propanol (4:1). The aqueous layer is extracted with additional CHCl₃/2-propanol (4:1) and the combined organic layers are dried over MgSO₄, filtered, and concentrated under reduced pressure. The resultant yellowish oil is purified by column chromatography (SiO₂, ethyl acetate) to give 3-[6-(4-trifluoromethoxyphenyl-amino)-pyrimidin-4-yl]-benzamide as a white solid. MSm/z 375.10(M+1).

GNF-2 Binds to the C-Terminal Myristate Pocket of Abl

To support the proposition that GNF-2 binds to the myristate binding pocket located at the Abl carboxy-terminus, an N-myristoylated peptide corresponding to the N-terminal amino acids 2-16 of c-Abl 1b has previously been demonstrated to displace Abl from a GNF-2 affinity matrix. Introduction of mutations to residues located at the entry (A337N) and at the back (A344L) of the myristoyl cleft conferred resistance to GNF-2 but not to imatinib has also been demonstrated. (Adrian et al., Nat. Chem. Biol. 2:95-102 (2006)).

To establish the binding site of GNF-2 to Abl by an independent biophysical method, nuclear magnetic resonance spectroscopy (NMR) was used to examine binding of GNF-2 to the Abl/imatinib complex. Ligand binding will cause chemical shift perturbations in the vicinity of the binding pocket. Using a fully assigned HSQC spectrum obtained with ¹⁵N-labeled Abl complexed with unlabeled imatinib, GNF-2 was shown to induce chemical shift changes that cluster around the myristate binding pocket. No significant chemical shift perturbations were observed for the ATP pocket, indicating that GNF-2 does not interfere with imatinib for binding at the ATP site. Myristic acid was found to induce qualitatively the same pattern of chemical shift perturbations, providing additional evidence that GNF-2 and myristate share the same binding site.

NMR studies, in which the chemical shift of a methyl group close to the myristate pocket of Abl was followed when titrating GNF-2 into the protein, revealed a dissociation constant of 0.5±0.1 μM for GNF-2 to the imatinib/Abl complex using the full-length catalytic domain (residues 229-515; Abl 1a numbering), and of 7.4±1.5 μM using the Imatinib/Abl complex with a C-terminal truncated form of Abl (residues 229-500) not including helix I. The lower affinity to the latter construct is probably due to helix I, which lines the myristate pocket, being involved in interactions with GNF-2. It is therefore shown that the binding site of GNF-2 to Abl is the myristate pocket

GNF-2 Binds to T315I Abl

Although the T315I “gatekeeper” mutation located in the ATP-binding cleft of BCR-ABL confers resistance to GNF-2 in cellular assays, this mutation is not expected to block binding of GNF-2 to the myristate pocket. We performed NMR-based titration experiments with T315I Abl (residues 229-500, not including helix I), and demonstrated that GNF-2 binds to this Abl mutant, albeit with a two-fold reduced affinity of 13.5±1.8 μM.

The binding of GNF-2 to the myristoyl pocket of Abl was further confirmed by crystallography. The structure of the Abl/imatinib/GNF-2 complex was obtained by soaking crystals of Abl/imatinib/myristate, obtained as described by Nagar et al. (Cell 112, 859-71 (2003)) in an excess of GNF-2. Based on the shape of the electron density, GNF-2 replaces the myristoylated peptide in the crystals. There are two molecules in the asymmetric unit and the myristate site is fully occupied in one and partially in the other.

GNF-2 binds in an extended conformation in the myristate pocket with the tri-fluoromethyl group buried at the same depth as the final two carbons of the myristate ligand. There is a favorable, but probably weak, polar interaction between one fluorine atom and the main chain of L340 (similar to that observed between nilotinib and Asp381 of Abl) and there are water mediated hydrogen bonds, but no direct hydrogen bonds with the protein. A water molecule forms a hydrogen bond bridge between the aniline NH and the main chain carbonyls of A433 and E462 in both the fully and partially occupied myristate binding sites in the crystal. As expected, the majority of the interactions between GNF-2 and the protein are hydrophobic. Residues contacting GNF-2 at the base of the pocket are L341 and A344 from αE, I432 from αF, V468 from αH, F493 from αI and I502 from αI′. The surface in the central part of the pocket is formed by A337 from αE, C464 and P465 from the start of αH, A433 from αF, and V506 from αI′. There are fewer interactions at the mouth of the pocket (Y435 from αF, E462 from the loop before αH and L510 at the end of αI′), which is reflected in the weak electron density and hence flexibility of the benzamide part of GNF-2. The mutation of three of these residues (C464Y, P465S and V506L) is found to cause resistance to the binding of GNF-2, presumably for steric reasons. The other two mutations found in this region (F497L and E505K) are in the second shell of residues forming the binding site, and are likely to have an indirect unfavorable steric effect.

The overall structure of the Abl kinase domain is similar to that of the myristate complex, except for the positions of residues between F497 and 5501 which are shifted by up to 4 Å. This is due to crystal contacts between this part of the structure and a neighboring molecule in the crystal. It does not have any affect on the myristate binding site, but changes the SH2-docking surface such that there would be a clash with helix αA of the SH2 domain. There is also a very small rotation of the N-terminal lobe of the kinase with respect to the C-terminal lobe, but this may also be due to slight changes in crystal packing due to the replacement of the myristoylated peptide. There is very little difference between the relative orientations of these lobes when comparing the Abl/imatinib complex with the Abl/imatinib/myristate complex. Also, there are no changes in the ATP site in any of these structures, which all have imatinib bound.

Furthermore, a systematic evaluation of the structural features that impart activity as a cellular BCR-ABL inhibitor was investigated in the context of binding to the myristate binding site. These studies revealed that the pyrimidine C4-position tolerated a variety of substituents, with cellular potency as a BCR-ABL inhibitor achieved with either meta- or para-substituted phenyls, such as meta-carboxamido (3-CONH₂ (GNF-2), 3-CONH(CH₂)₂OH (GNF-5), sulfones (SO₂CH₃) and sulfonamides (SO₂NHR).

Although the mechanism for activity is not required to practice the invention, one interesting observation was the 4,6-substitution requirement for the central pyrimidine was different from that of ATP-competitive kinase inhibitors, where the 2,4-pyrimidine is generally favored as a motif that binds to the kinase hinge region. This is believed to be a consequence of the ability of 2,4-pyrimidines to assume the cis-conformation with respect to the NH—C2 bond and the ability of this conformation to form a bidentate H-bond to the kinase hinge region. Considering that compounds of the GNF-2 series were not ATP-competitive BCR-ABL inhibitors, the importance of the 4,6-pyrimidine may be a consequence of a trans-configuration for activity. To investigate this hypothesis, a series of structurally similar compounds were prepared which would exhibit different preferences for cis- or trans-conformations due to potential steric interactions between the ortho-position of the 4-trifluoromethoxyaniline ring and the pyrimidine-C5 in the cis-conformation. Evaluation of the BCR-ABL activity of these compounds demonstrated that only those with a preference for the trans-conformation were active (Table 4). The trans-conformation is also consistent with the subsequently determined co-crystal structure which demonstrates that this conformation is required to accommodate the ligand in the narrow hydrophobic myristate binding site.

TABLE 4

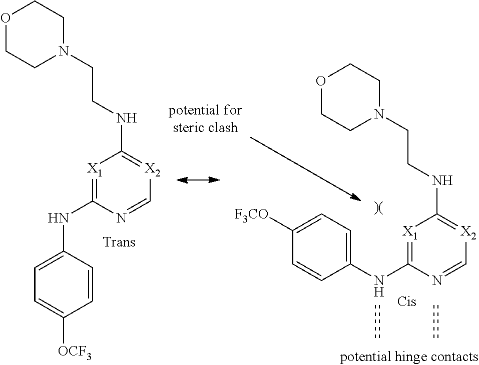

| X₁ | X₂ | IC₅₀ Bcr-Abl (nM) | Cis | Trans |
|---|---|---|---|---|
| CH | N | 810 | Disfavored | Favored |
| N | CH | >10,000 | Favored | Disfavored |
| N | N | >10,000 | Equal | Equal |
| CCH₃ | N | 970 | Disfavored | Favored |

GNF-2 and Imatinib Combinations Reduce the Emergence of Clones Expressing Drug-Resistant Mutant Forms of BCR-ABL

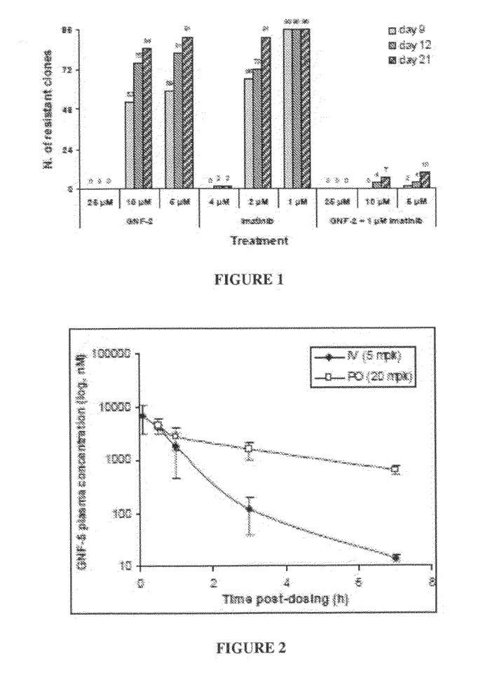

BCR-ABL transformed Ba/F3 cells (Ba/F3.p210) can develop resistance to imatinib as the result of point mutations that reduce the affinity of imatinib for the active site and recapitulate many of the clinically observed mutations. Consistent with the ability of GNF-2 and imatinib to bind to BCR-ABL simultaneously, it was previously demonstrated that combinations of the two compounds can inhibit BCR-ABL-dependent proliferation synergistically. We sought to investigate the frequency with which BCR-ABL dependent Ba/F3 cells would become resistant to combinations of GNF-2 and imatinib compared to each compound alone. Incubation of Ba/F3.p210 cells with 1 μM imatinib or 5 or 10 μM GNF-2 resulted in the emergence of resistant colonies by day nine (FIG. 1). The number of clones that were resistant to 1 μM imatinib was reduced by 98 and 100% when combined with 5 and 10 μM of GNF-2 respectively by the ninth day and 90-92% by day 21. These results demonstrate that combinations of GNF-2 and imatinib can cooperate to suppress the emergence of resistance mutations.

To identify the mutations emerging from the combination treatment, we partially sequenced the cDNA coding for BCR-ABL (kinase fragments Q108-D325 and W430-R564) in the resistant clones. Sequence analysis revealed the presence of different single point mutations in 59% of the clones resistant to GNF-2/imatinib combinations. Of these, the F317L and Q252H substitutions have previously been reported to confer resistance to imatinib. However, P465S, E505K and F497L were also identified, which are located in the myristate-binding pocket and are believed to sterically interfere with the ability of GNF-2 to bind to this site.

### Example 3

### N-(2-hydroxyethyl)-3-(6-(4-(trifluoromethoxy)phenylamino)pyrimidin-4-yl)benzamide (GNF-5)

To a solution of 3-[6-(4-trifluoromethoxy-phenylamino)-pyrimidin-4-yl]-benzoic acid (81 mg, 0.22 mmol), ethanolamine (16 mg, 0.26 mmol), and di-isopropylethylamine (84 mg, 0.65 mmol) in DMF (0.5 mL) is added 2-(1H-7-azabenzotriazol-1-yl)-1,1,3,3-tetramethyluranium (HATU) (99 mg, 0.26 mmol) at room temperature. The reaction mixture is stirred for 4 hrs at rt and purified by flash column chromatography (SiO₂, CH₂Cl₂/MEoh (v/v)=20/1) to give N-(2-hydroxyethyl)-3-(6-(4-(trifluoromethoxy)phenylamino)pyrimidin-4-yl)benzamide as a white solid. MS m/z 419.2 (M+1).

GNF-5 Pharmacokinetic Parameters in Mice

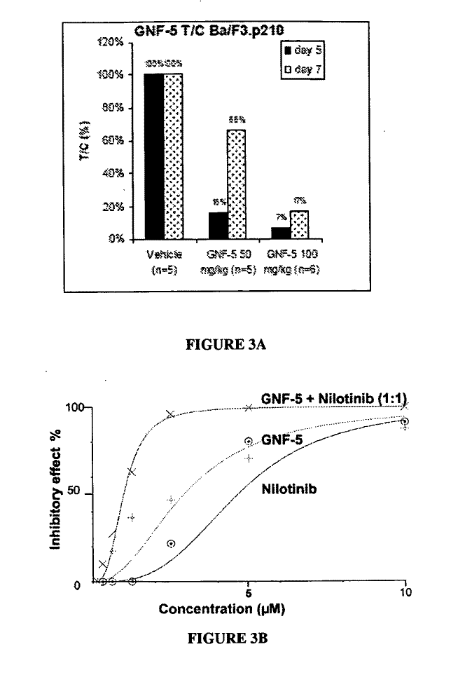

Following an oral dose of 20 mg/kg to Balb-C mice, GNF-5 appeared rapidly in the circulation to attain a maximum concentration of 4.4±1.3 μM at 0.5 h, decreasing to 0.63±0.12 μM after 7 hours, with a terminal half-life of 2.3 hours (FIG. 2); oral bioavailability was 44.8±7.5%. In SCID mice, as used for efficacy studies in the xenograft model, the pharmacokinetic profile in plasma was similar to that observed in normal mice.

Pharmacokinetic parameters are shown in Table 5: AUC=area under the curve (measure of exposure), Cₘₐₓ=maximum plasma concentration, Tₘₐₓ=time of maximum plasma concentration, Cₗₐₛₜ=concentration at last measured time point, T_{1/2}=time required for plasma concentration to reach half of the highest concentration, Vₛₛ=volume of distribution, F=percentage oral bioavailability.

TABLE 5

| | |
|---|---|
| AUC_inf (min * ug/mL) | 292.37 ± 49.18 |
| AUC_inf (hrs * nM) | 11647 ± 1959 |
| Cmax (nM) | 4366.08 ± 1344.67 |
| Tmax (hrs) | 0.50 ± 0.00 |
| Clast (nM) | 636.16 ± 121.20 |
| T½ (hrs) | 2.30 ± 0.10 |
| Vss (L/kg) | 9.18 ± 1.82 |
| F (%) | 44.82 ± 7.54 |

GNF-5 Displays In Vivo Efficacy in a Mouse Xenograft Model of CML

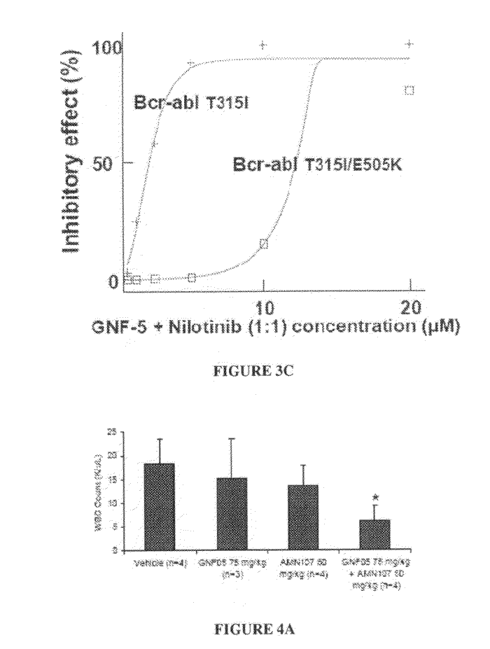

We evaluated the antitumor activity of GNF-5 in vivo in a murine myeloproliferative disease model. Disease was established in SCID beige mice by inoculation of Ba/F3 cells engineered to express wild-type p210 BCR-ABL and firefly luciferase, such that disease burden could be assessed after luciferin injection by non-invasive imaging using a Xenogen IVIS™ system. As expected, animals within a control group that received no drug treatment showed a continuous increase in tumor burden. In contrast, oral treatment with either 50 or 100 mg/kg of GNF-5 administered twice daily resulted in a substantial dose-response reduction in tumor burden within the first week. Responses approaching stasis were achieved by the fifth day post-treatment with 50 or 100 mg/kg b.i.d. of GNF-5, with tumor/control (T/C) of 16 and 7% respectively. Responses were maintained within the 100 mg/kg group (T/C=17%) at day 7; however, the initial response to the 50 mg/kg b.i.d. regime was followed by a relapse (T/C of 66%) (FIG. 3A).

To examine target modulation in vivo, Ba/F3.p210-bearing mice were treated with a single dose of GNF-5 (100 mg/kg) or vehicle. Bone marrows were harvested after 3, 7 and 24 hours and were analyzed by flow cytometry for phospho-Stat5 levels using a specific phospho-Y694 antibody. Significant reduction in pStat5 levels was observed within 7 hours after delivery of a single dose of GNF-5. Phosphorylation of Stat5 was notably inhibited, indicating blockade of downstream BCR-ABL signaling. These results demonstrate that inhibition of BCR-ABL dependent cellular proliferation can be achieved in vivo at well-tolerated doses but that a residual population of cells survives the treatment.

GNF-5 and Nilotinib Combinations can Inhibit T315I BCR-ABL-Dependent Proliferation

GNF-5 maintains cellular potency against E255V (IC₅₀=380 nM) and M351T mutants (IC₅₀=930 nM), but is significantly less active against the G250E (IC₅₀=4.52 μM), F317L (IC₅₀>10 μM) and T315I (IC₅₀>5 μM) mutants. Nilotinib exhibits potent activity against all these mutants with the exception of T315I (IC₅₀>10 μM)⁵. Encouraged by NMR and isothermal calorimetry experiments which demonstrated that GNF-2 and imatinib could bind to wild-type and T315I Abl simultaneously (data not shown), we tested the combined effects of GNF-5 and nilotinib on T315I BCR-ABL dependent cellular proliferation and inhibition of phosphorylation of the downstream substrate Stat5 by fluorescent activated cell sorting (FACs). The proliferation assays demonstrated greater than 50% inhibition of T315I BCR-ABL-dependent cell growth could be achieved at a range of GNF-5 and nilotinib concentrations with a calculated combination index¹⁵ (CI) of 0.6 indicating moderate synergy (FIG. 3B). For example, at a fixed nilotinib concentration of 20 μM, GNF-5 inhibits T315I BCR-ABL-dependent proliferation with an IC₅₀ of 0.76 μM

We also demonstrated by flow cytometry analysis that GNF-5 and nilotinib act additively to inhibit Stat5 phosphorylation. For example, while 10 μM nilotinib or 1 μM GNF-5 alone have no effect on T315I BCR-ABL-mediated Stat5 phosphorylation, the combination of the two compounds results in a significant inhibition of Stat5 phosphorylation that can be rescued by the addition of IL-3 to the medium. We confirmed that the cooperativity observed between GNF-5 and nilotinib is directly mediated by inhibition of BCR-ABL based upon the ability of a double mutation of T315I in the ATP-site and E505K in the myristate site to confer complete resistance to the combination of both inhibitors (FIG. 3C).

GNF-5 and Nilotinib Combinations Exhibit In Vivo Efficacy Against T315I BCR-ABL

A bone marrow transduction/transplantation mouse model more closely resembling human CML disease was used to demonstrate the in vivo efficacy of GNF-5 on wild-type and T315I BCR-ABL (see, Roumiantsev et al., Proc Natl Acad Sci USA 99, 10700-5 (2002). In an initial experiment, bone marrow cells from 5-fluorouracil (5-FU) pretreated donor mice were transduced with a wild type BCR-ABL retroviral vector and transplanted into irradiated recipient mice. Seven days after transplantation, a dose regimen consisting of 50 mg/kg GNF-5 twice daily or vehicle was administered during seven days. Peripheral blood cell counts measured on the last day of treatment were high, with 95% neutrophils or blast cells, in the vehicle treated mice consistent with development of CML-like disease. In contrast, GNF-5 treated mice showed normal blood cell counts. The spleen size from the vehicle group was increased 3- to 4-fold compared to those of normal mice (normal spleen weight: 80-90 mg), while GNF-5 treated mice had normal spleen weights.

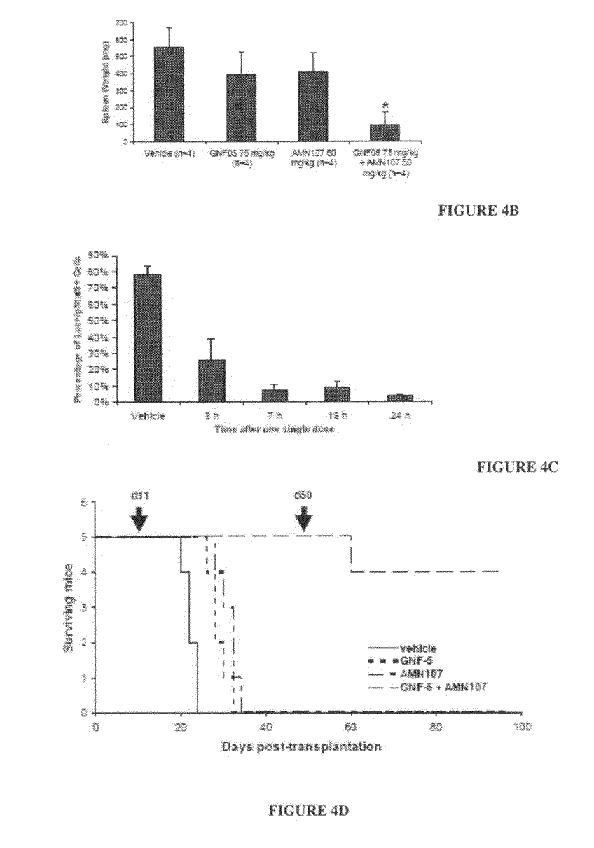

To assess whether the combination of GNF-5 with nilotinib would result in efficacy in a T315I BCR-ABL mutant (luciferase) bone marrow transplantation model, treatment was started fifteen days after transplantation with a b.i.d. dosing regimen of nilotinib 50 mg/kg or GNF-5 75 mg/kg alone or in combination. Seven days after treatment was initiated, the spleen size and blood cell counts in the different dosing groups were measured. Mice treated with either nilotinib or GNF-5 alone showed no significant response compared to the vehicle group, with 2-3 fold higher cell counts and spleens four-fold larger than those of healthy mice. The combination of both compounds normalized blood cell counts and spleen size without signs of toxicity (FIGS. 4A and 4B), suggesting an additive effect of the compounds in a combination treatment.

To establish an efficacy/pharmacodynamic response correlation, bone marrow cells from the different mice groups were isolated at the end of the efficacy study and stained with anti-p-Stat5 and anti-luciferase specific antibodies. The number of p-Stat5 positive cells within the luciferase gate was quantified by flow cytometry. The percentage of p-Stat5 positive BCR-ABL expressing bone marrow cells was similar (approx. 25%) in the vehicle, GNF5 and nilotinib treated groups. In the combination group, the percentage of p-Stat5 positive cells was about 6%, reflecting a correlation between the tumor growth inhibition and BCR-ABL signaling blockade. To determine the extent of the inhibition, mice were treated with a single dose of the combination (50 mg/kg nilotinib plus 75 mg/kg GNF-5) or vehicle and the bone marrow cells collected 3, 7, 16 and 24 hours post-dose and double stained with anti-luciferase and anti-pStat5 antibodies. In the vehicle group, about 80% of the luciferase positive cells had phosphorylated Stat5. Three hours after dosing, Stat5 phosphorylation was reduced from 80% to 25% and, from 7 to 24 h, the number of pStat5 positive cells remained below 10%, demonstrating a strong and sustained inhibition of BCR-ABL mediated signaling following administration of the GNF-5/nilotinib combination (FIG. 4C).

In a third experiment, the survival of the mice transplanted with T315I BCR-ABL transduced bone marrow and treated with GNF-5, nilotinib or a combination of both compounds was monitored. Mice transplanted with T315I BCR-ABL transduced bone marrow and treated with vehicle control died by day 24 after transplantation, with a median survival of 22 days (FIG. 4D). GNF-5 (75 mg/kg b.i.d.) extended survival (median 28 days) significantly compared to vehicle treated controls (P=0.023). Mice treated with nilotinib alone (50 mg/kg b.i.d.) also survived longer (median 32 days) than those treated with vehicle (P=0.023). The overall survival of mice treated with GNF-5 plus nilotinib was improved compared to those treated with either GNF-5 (P=0.002) or nilotinib alone (P=0.002), with all the mice surviving by day 50 post-transplantation, after which the treatment was discontinued. Forty six days after the combination treatment was completed, 4 out of 5 mice survived without showing signs of disease. Cumulatively, these results suggest that a combination of an ATP-competitive with an allosteric inhibitor may be a therapeutically appropriate strategy to target the T315I BCR-ABL mutation.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, polynucleotide and polypeptide sequence accession numbers and other documents cited herein are hereby incorporated by reference in their entirety and for all purposes to the same extent as if each of these documents were individually so denoted.

1. A composition comprising a combination of an ATP-competitive BCR-ABL inhibitor and an ATP non-competitive BCR-ABL inhibitor;
wherein said ATP-competitive BCR-ABL inhibitor is 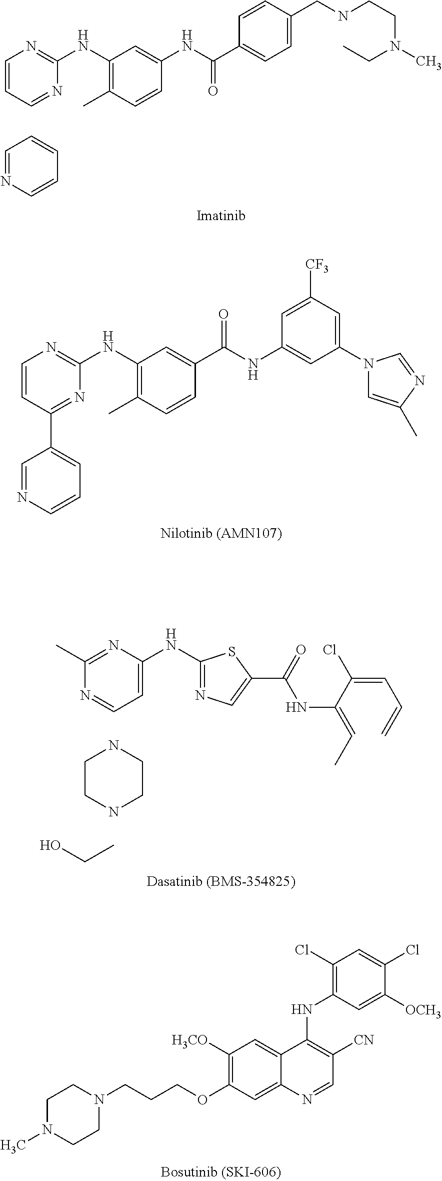 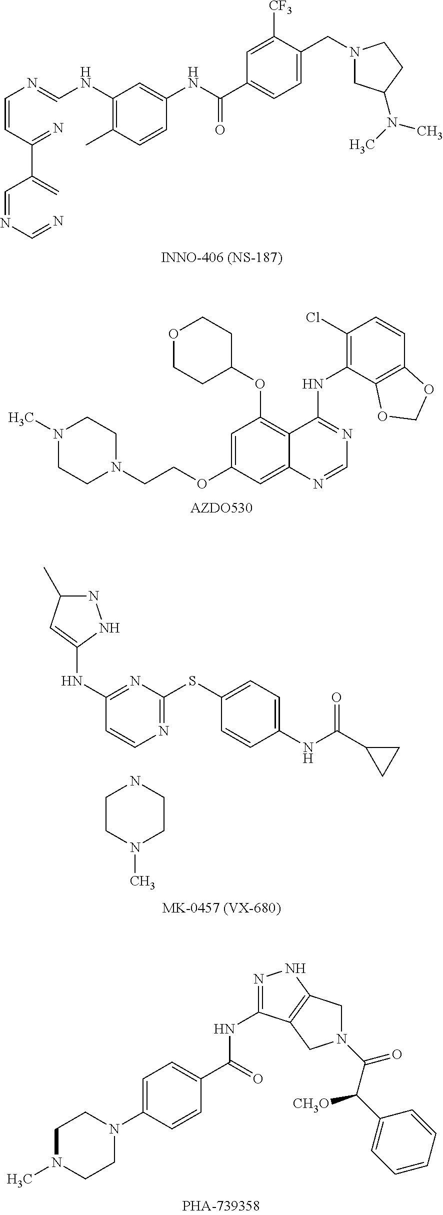 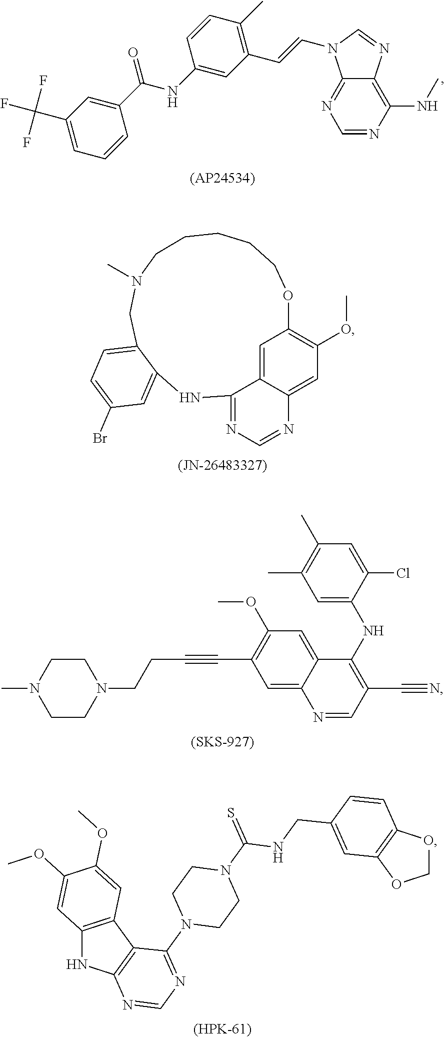
AT-9283 (Astex Therapeutics), EXEL-2280 (Exelisis), or TG-100572 (TargeGen); and
wherein said ATP non-competitive BCR-ABL inhibitor is a compound of Formula (1): 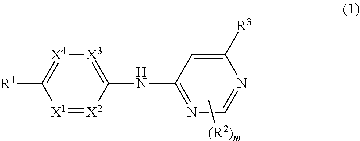
or a pharmaceutically acceptable salt thereof;
wherein X¹, X², X³ and X⁴ are each CH; or one of X¹, X², X³ and X⁴ is N and the others are CH;
R¹ is OCF₃ or CF₃;
R² is C₁₋₆ alkyl;
R³ is NR(CH₂)₂NR⁴R⁵ or a 5-7 membered heterocyclic ring; or R³ is aryl or a 5-7 membered heteroaryl, each of which is optionally substituted with 1-2 R⁶ groups or optionally substituted with an aryl or heteroaryl, each of which is optionally substituted with 1-2 R^{6a} groups; wherein R⁶ and R^{6a} are independently CONR(CH₂)ₙOR⁷, CONR(CH₂)ₙNR⁴R⁵, CONR⁴R⁵, NR(CH₂)ₙOR⁷, NR(CH₂)ₙNR⁴R⁵, SO₂NRR⁷, NR⁴R⁵ or SO₂R⁸;
R⁴ is H or C₁₋₆ alkyl;
R⁵ is H, C₁₋₆ alkyl, aryl or heteroaryl;
alternatively, R⁴ and R⁵ together with N in NR⁴R⁵ may form a 5-7 membered ring;
R and R⁷ are independently H or C₁₋₆ alkyl;
R⁸ is C₁₋₆ alkyl;
m is 0-1; and
n is 1-4;
provided said ATP-competitive inhibitor is not imatinib when said non-ATP competitive inhibitor is

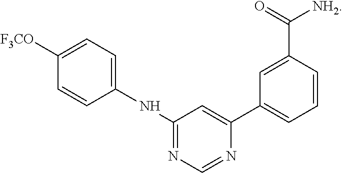

2. The composition of claim 1, wherein said non-ATP competitive inhibitor binds to the myristate binding site of BCR-ABL.

3. The composition of claim 1, wherein said non-ATP competitive inhibitor is a compound of Formula (2): 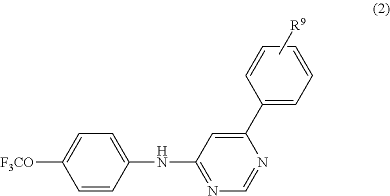
wherein R⁹ is in the meta or para-position, and is carboxamido, CONH(CH₂)₂OH, sulfones (SO₂CH₃) or sulfonamides (SO₂NHR).

4. The composition of claim 1, wherein X¹, X², X³ and X⁴ in Formula (1) are each CH.

5. The composition of claim 1, wherein R¹ in Formula (1) is OCF₃.

6. The composition of claim 1, wherein R³Formula (1) is morpholinyl, imidazolyl or pyridyl, wherein said pyridyl is optionally substituted with 1 R^{6a} group; and R^{6a} is as defined in claim 1.

7. The composition of claim 1, wherein R³ is phenyl optionally substituted in the meta- or para-position with 1 R⁶ group; and R⁶ is as defined in claim 1.

8. The composition of claim 1, wherein R³ in Formula (1) is NR(CH₂)₂NR⁴R⁵, and R⁴ and R⁵ together with N form morpholinyl.

9. The composition of claim 1, wherein said compound of Formula (1) is selected from the group consisting of:

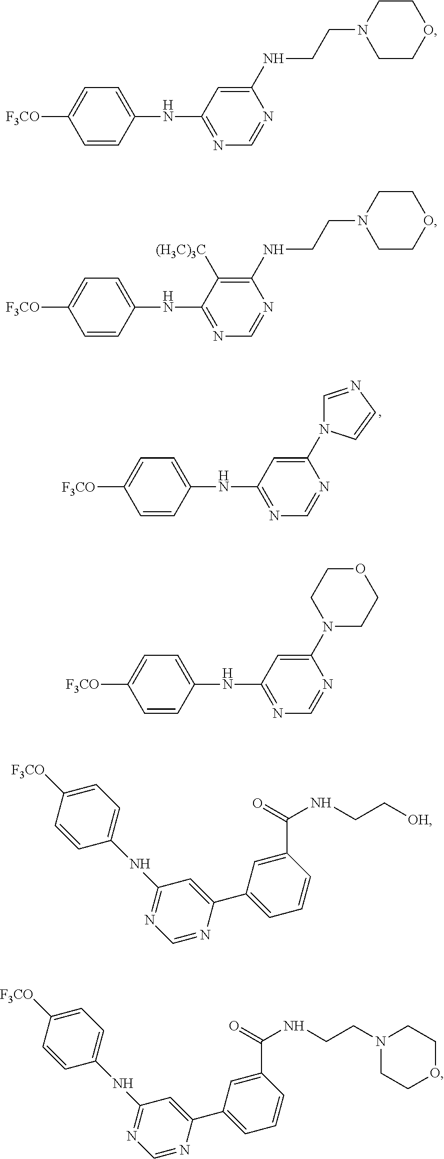

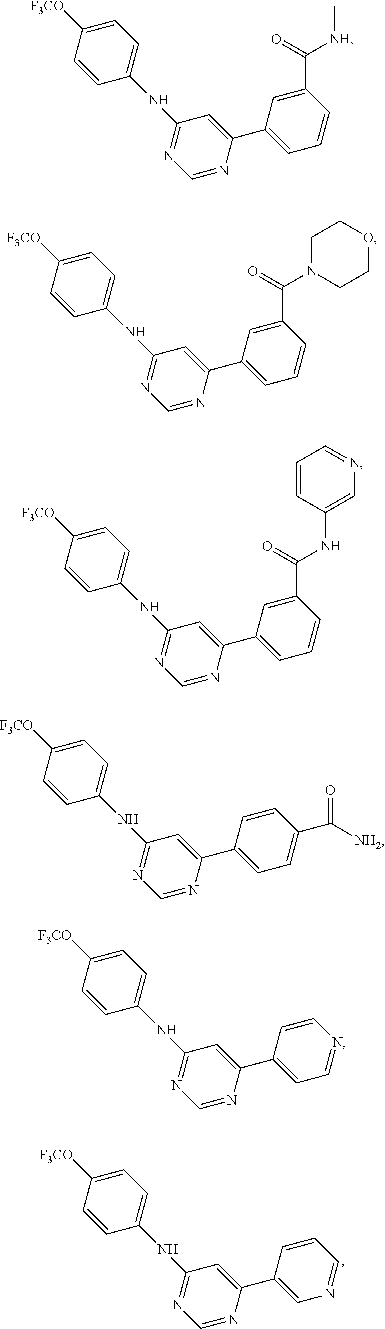

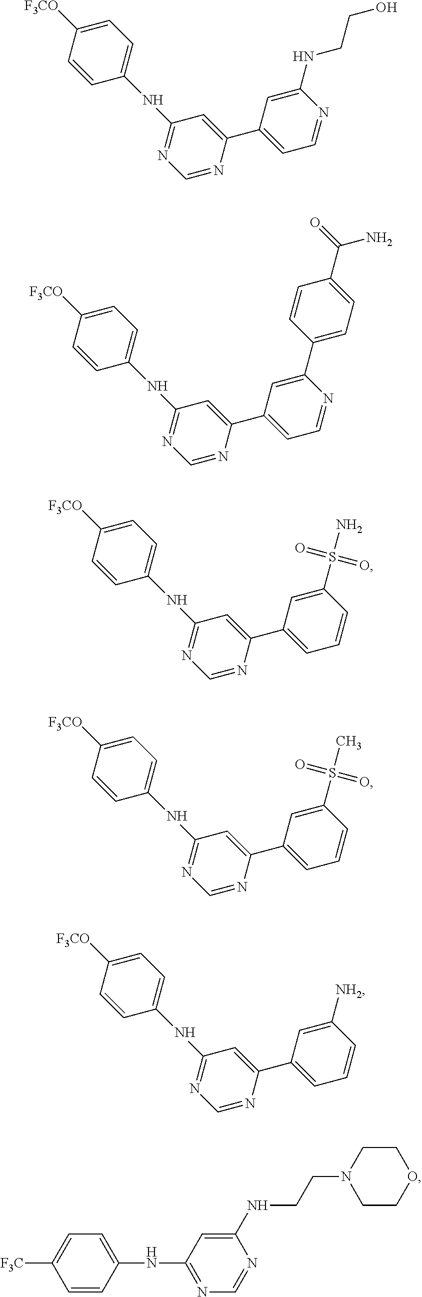

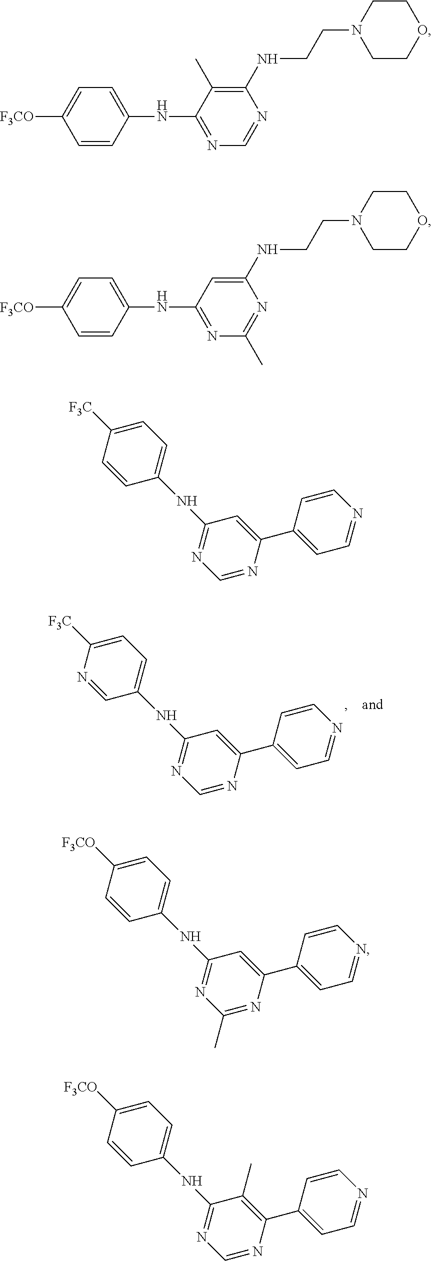

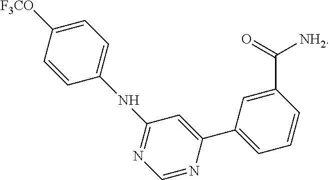

10. The composition of claim 9, wherein said compound of Formula (1) is

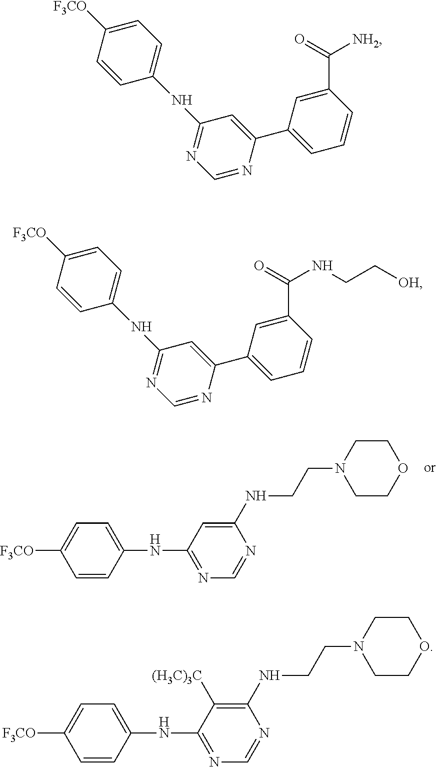

11. The composition of claim 1, wherein said ATP-competitive BCR-ABL inhibitor is imatinib, nilotinib or dasatinib.

12. The composition of claim 1, wherein said ATP-competitive BCR-ABL inhibitor is nilotinib and said compound of Formula (1) is

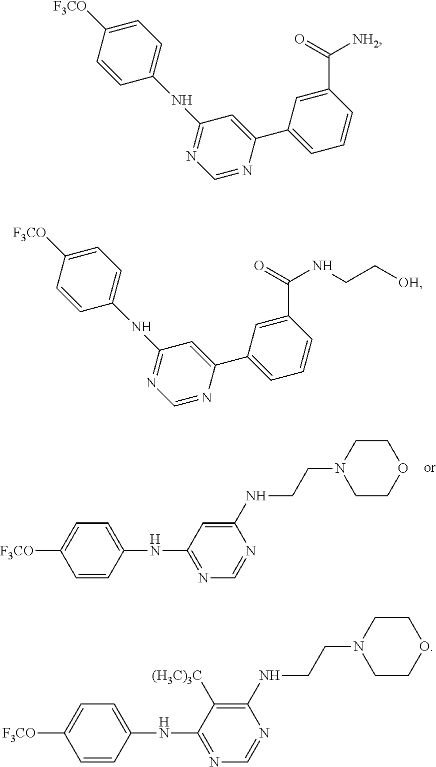

13. A method for treating a BCR-ABL positive leukemia, comprising administering to a cell or a subject, a therapeutically effective amount of a composition comprising an ATP-competitive BCR-ABL inhibitor and an ATP non-competitive BCR-ABL inhibitor;
wherein said ATP-competitive BCR-ABL inhibitor is 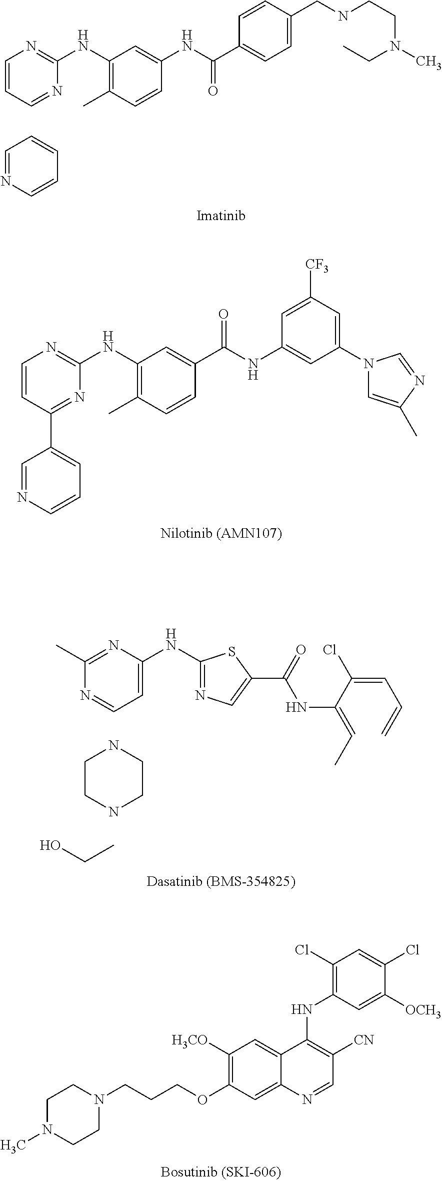 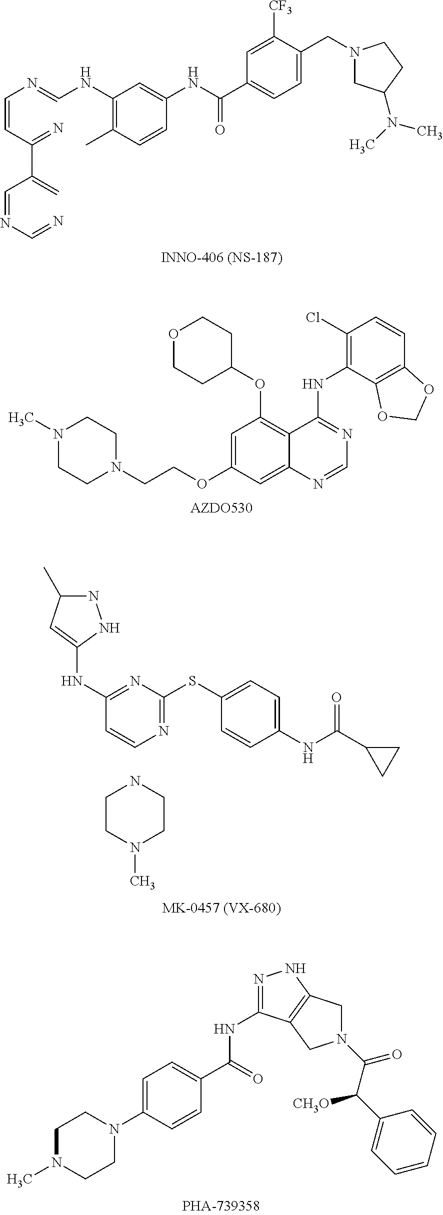 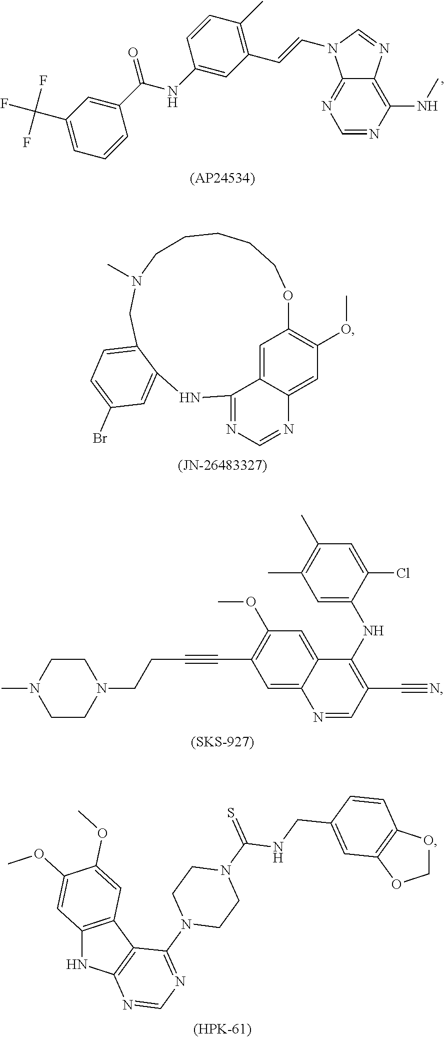
AT-9283 (Astex Therapeutics), EXEL-2280 (Exelisis), or TG-100572 (TargeGen); and
said ATP non-competitive BCR-ABL inhibitor is a compound of Formula (1): 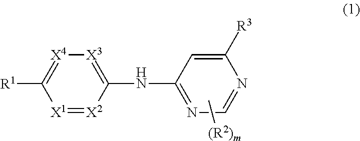
or a pharmaceutically acceptable salt thereof;
wherein X¹, X², X³ and X⁴ are each CH; or one of X¹, X², X³ and X⁴ is N and the others are CH;
R¹ is OCF₃ or CF₃;
R² is C₁₋₆ alkyl;
R³ is NR(CH₂)₂NR⁴R⁵ or a 5-7 membered heterocyclic ring; or R³ is aryl or a 5-7 membered heteroaryl, each of which is optionally substituted with 1-2 R⁶ groups or optionally substituted with an aryl or heteroaryl, each of which is optionally substituted with 1-2 R^{6a} groups; wherein. R⁶ and R^{6a} are independently CONR(CH₂)ₙOR⁷, CONR(CH₂)ₙNR⁴R⁵, CONR⁴R⁵, NR(CH₂)ₙOR⁷, NR(CH₂)ₙNR⁴R⁵, SO₂NRR⁷, NR⁴R⁵ or SO₂R⁸;
R⁴ is H or C₁₋₆ alkyl;
R⁵ is H, C₁₋₆ alkyl, aryl or heteroaryl;
alternatively, R⁴ and R⁵ together with N in NR⁴R⁵ may form a 5-7 membered ring;
R and R⁷ are independently H or C₁₋₆ alkyl;
R⁸ is C₁₋₆ alkyl;
m is 0-1;
n is 1-4;
provided said ATP-competitive inhibitor is not imatinib when said non-ATP competitive inhibitor is

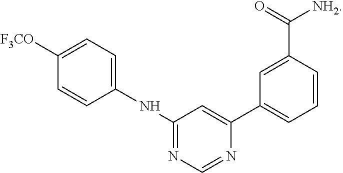

14. The method of claim 13, wherein said ATP-competitive inhibitor and non-ATP competitive inhibitor exhibits a synergistic effect.

15. The method of claim 13, wherein said BCR-ABL positive leukemia is chronic myeloid leukemia or acute lymphocyte leukemia.

16. The method of claim 13, wherein said non-ATP competitive inhibitor binds to the myristate binding site of BCR-ABL.

17. The method of claim 13, wherein said compound of Formula (1) is selected from the group consisting of:

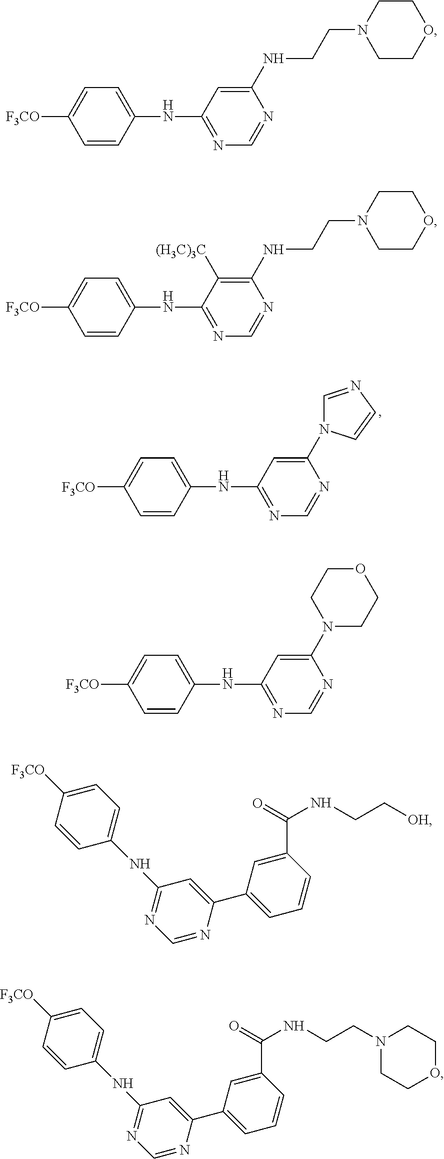

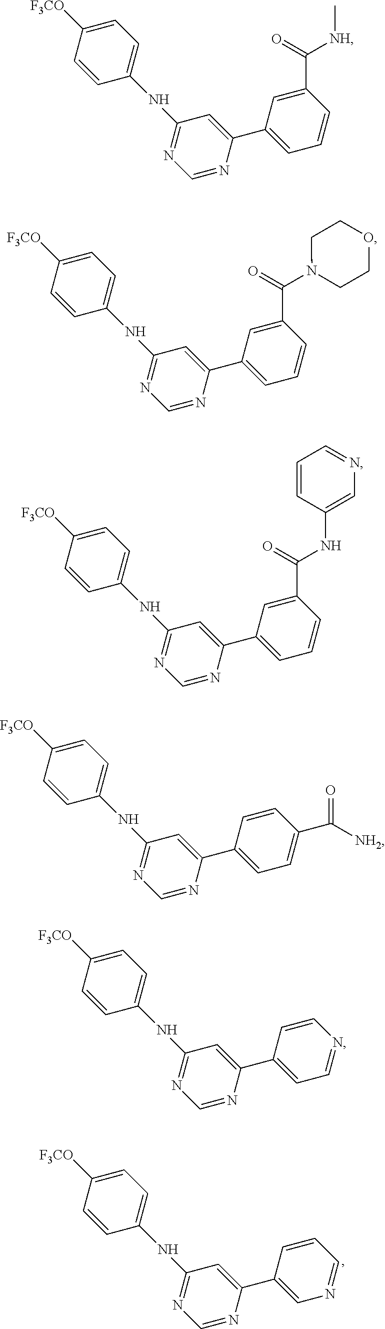

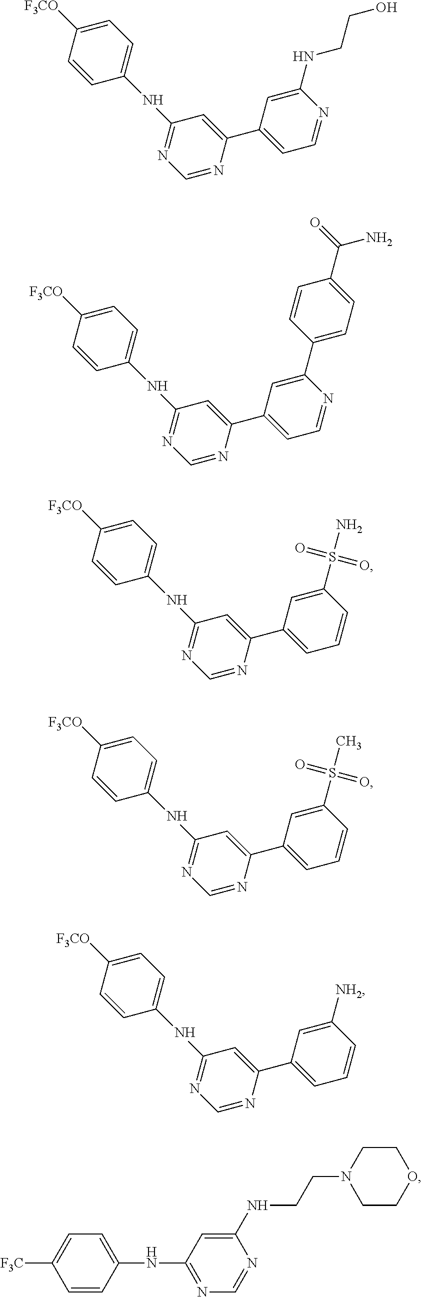

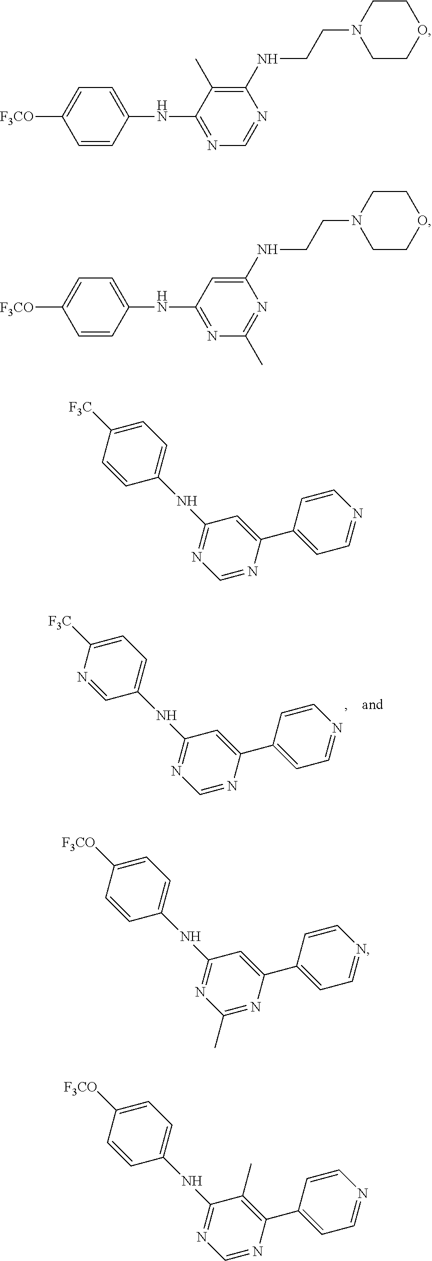

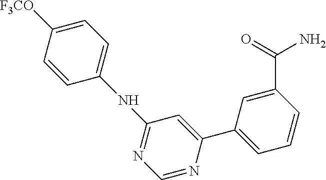

18. The method of claim 17, wherein said compound of Formula (1) is

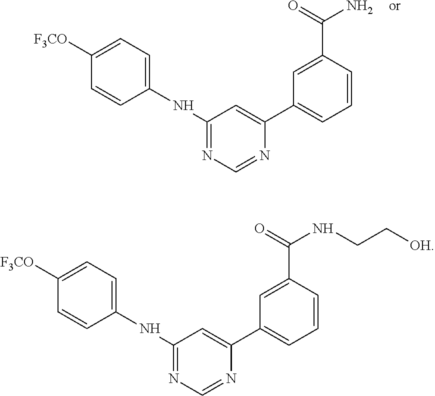

19. The method of claim 13, wherein said ATP-competitive BCR-ABL inhibitor is imatinib, nilotinib or dasatinib.

20. The method of claim 13, wherein said ATP-competitive BCR-ABL inhibitor is nilotinib and said compound of Formula (1) is

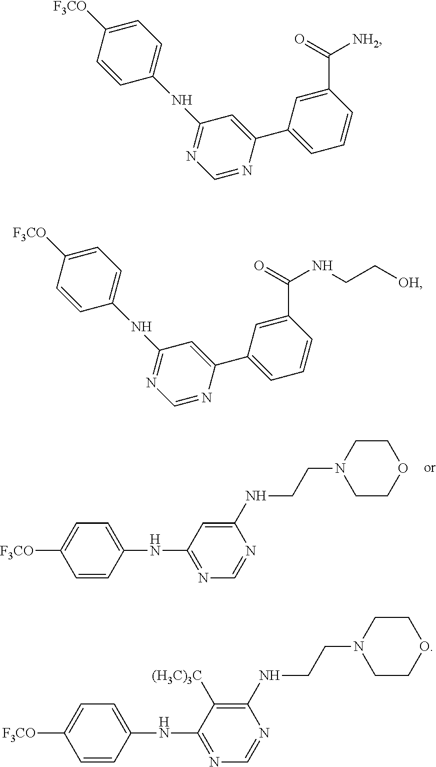

21-22. (canceled)
